(19) United States
(12) Patent Application Publication
(10) Pub. No.: US 2023/0090884 A1
(43) Pub. Date: Mar. 23, 2023

(54) DEVICE AND METHODS FOR ISOLATING EXTRACELLULAR MATRIX BODIES

(71) Applicant: AUFBAU MEDICAL INNOVATIONS LIMITED, Dublin (IE)

(72) Inventors: John T. G. PENA, New York, NY (US); James Murray MITCHELL, Danville, CA (US); Luca D'AGOSTINO, Ithaca, NY (US); Sangeetha Tandalam PALANIVELU, Ithaca, NY (US); Kunihiro URYU, Ithaca, NY (US); Harmon Lawrence REMMEL, New York, NY (US)

(73) Assignee: AUFBAU MEDICAL INNOVATIONS LIMITED, Dublin (IE)

(21) Appl. No.: 17/934,675

(22) Filed: Sep. 23, 2022

Related U.S. Application Data
(63) Continuation of application No. PCT/US2021/023827, filed on Mar. 24, 2021.
(60) Provisional application No. 62/994,469, filed on Mar. 25, 2020.

(51) Int. Cl. B01L 3/00

(52) U.S. Cl. CPC B01L 3/502761; B01L 3/502715; B01L 3/502746; B01L 3/502753; B01L 2300/0816; B01L 2300/0867; B01L 2200/027; B01L 2200/0636; B01L 2300/0861

(57) ABSTRACT

Devices, methods and systems are provided for isolating extracellular matrix bodies. More particularly, this invention discloses devices, methods and systems for isolating extracellular matrix bodies from a biological sample for use in diagnosis and prognosis of a subject. A device may have restriction channels for isolating extracellular matrix bodies and uniform flow channels for precise pressure measurements.

### TECHNICAL FIELD

This invention relates to devices, methods and systems for isolating extracellular matrix bodies. More particularly, this invention discloses devices, methods and systems for isolating extracellular matrix bodies from a biological sample for use in diagnosis and prognosis of a subject.

### BACKGROUND

Conventional methods for diagnosis and prognosis of disease can require analyzing or isolating a tiny fraction of a biological sample. A corresponding analysis of components from the tiny fraction may be used in diagnosis and prognosis of disease. For example, individual cells, and even individual nucleic acid molecules can be detected and analyzed. However, a drawback of such conventional methods is to rely on a very small amount of biological material, and correspondingly small signal, for decision-making. Useful signals may be lost in other biological materials that are not measured.

Conventional methods for diagnosis and prognosis of disease often require biological samples to be obtained and well-known structures to be isolated from the sample for further analysis. For example, intact organ tissues, whole cells, exosomes and other well-known structures may be isolated and characterized. Drawbacks of such methods include the inability to detect or characterize disease when the well-known structures do not readily reflect the disease state.

Additional drawbacks of conventional methods for diagnosis and prognosis of disease include the use of biomarkers which relate only to a particular well-known structural element of a biological sample of a subject, for example exosomes. In these methods, biomarkers are often inherently limited by not being related directly to a pathology of interest. Conventional methods often attempt to take into account a number of biomarkers that can be remotely or partially associated with a disease, with the hope that statistics will provide a diagnostic answer. Combinations of biomarkers are routinely required and results are highly unpredictable.

Further drawbacks of conventional devices include inability to precisely measure flow and pressure in complex fluids containing biological components. A fluids containing biological components may cause variation of flow and pressure because of interaction of the biological components with the device performing the measurement.

What is needed are devices and systems for isolating particles from a biological sample for purposes of diagnosis which can provide increased sample isolates that are relevant to a particular biology. More particularly, there is a need for devices, methods and systems for isolating significant components from a biological sample for use in diagnosis and prognosis of disease.

There is an urgent need for methods and devices for isolating particles from a biological sample that can isolate pertinent fractions of a biological material which correspond more closely to disease states, and measure differential pressure and flow in pertinent fluids.

### BRIEF SUMMARY

This invention provides devices and systems for isolating particles from a biological sample applicable to various diseases and conditions. Methods and systems for isolating particles from a biological sample are provided which can increase sample isolates relevant to biology. Devices and methods of this invention can be used for isolating significant components from a biological sample for use in diagnosis and prognosis of disease.

In some embodiments, the methods and devices of this invention for isolating particles from a biological sample can be used with various kinds of biological samples, including bodily fluids, tissues, and cells. In certain embodiments, methods of this disclosure can isolate relevant fractions of a biological material corresponding closely to disease states.

Methods and devices of this disclosure can be used for isolating a significant fraction of a biological sample, with analysis of its components related to diagnosis and prognosis of disease. In some embodiments, a significant amount of biological material, and correspondingly improved signal level, can be obtained for decision-making. Aspects of this invention include preserving the composition and properties of extracellular matrix bodies (EMB) as indicators for disease.

In some aspects, this invention provides enhanced ability to detect or characterize disease using extracellular matrix bodies, which may more readily reflect a disease state.

In additional aspects, devices and methods of this disclosure can provide increased signal for pertinent biomarkers which relate a specific biological fraction to a disease state. Biomarkers of this disclosure can be associated with a disease and provide a diagnostic tool.

A device of this disclosure can provide precise measurement of flow and pressure in a fluid containing biological components by maintaining a continuous and substantial flow in the device, so that a sensor can precisely measure differential pressure and flow. Devices and system disclosed herein can provide improved measures of flow and pressure in a fluid containing biological components by arrangements of channels that maintain continuous and substantial flow. In some embodiments, a device of this disclosure may have one or more channels that do not substantially restrict the flow of a fluid so that a continuous and substantial flow is maintained in the system.

Devices and systems of this disclosure can isolate a unique subpopulation or subset fraction of a biological sample. In some embodiments, a unique subset fraction of a biological sample can be associated with a disease. In certain embodiments, a unique subset fraction of a biological sample can be substantially composed of extracellular matrix bodies.

In further aspects, this disclosure provides devices and methods for isolating, detecting, and/or analyzing ultrastructural components of a fluid containing biological material or molecules. In certain embodiments, ultrastructural components may be associated with disease.

Embodiments of this invention can be used to isolate, extract, and utilize extracellular matrix bodies (EMB) that are a source of multiple and specific disease biomarkers.

This invention includes devices for isolating, detecting and analyzing compositions of extracellular matrix bodies, biological particles and complexes for various uses.

In certain aspects, extracellular matrix bodies can operate as biomarkers through their morphological features. In further aspects, extracellular matrix bodies can operate by containing isolated biochemical markers that may be presented in disease pathways.

Aspects of this invention can further provide a diagnostics system including devices for detecting and measuring biomarkers via disease-associated extracellular matrix bodies (EMB) and/or biological particles or complexes.

In further embodiments, this disclosure describes methods and devices for preparing and analyzing a sample of a biological material.

Samples of a biological material can include bodily fluids, tissues, and cells.

Embodiments of this invention include the following:

A device for isolating a fraction of a biological sample, comprising:

one or more restriction channels having an inlet end and an outlet end, wherein the inlet end and outlet end are in fluid communication through the channel;

a plurality of spaced-apart obstructions lodged in the restriction channels for providing resistance to flow, wherein the spacing between obstructions decreases in the direction from the inlet end to the outlet end; and

an inlet reservoir for holding a fluid, wherein the inlet fluid reservoir is in fluid communication with the inlet end of the restriction channels;

one or more uniform flow channels having an inlet end and an outlet end, wherein the inlet end and outlet end are in fluid communication through the channel, wherein the inlet end is in fluid communication with the inlet reservoir.

The device above, further comprising a pressure source for applying pressure to the fluid in the inlet reservoir; and/or a flow sensor in fluid communication with the inlet reservoir for measuring the flow rate and pressure of the fluid at the inlet reservoir.

The device may further comprise an outlet reservoir in fluid communication with the outlet ends of the restriction channels and uniform flow channels.

The device above, wherein the restriction channels comprise a barrier band having fenestrations of at least about 1 micrometers, or at least about 2 micrometers, or at least about 4 micrometers, or at least about 10 micrometers, or at least about 25 micrometers, or at least about 50 micrometers, or at least about 100 micrometers, or at least about 200 micrometers, or at least about 500 micrometers.

The device above, wherein the restriction channels comprise fenestrations of about 1-4 micrometers, or about 1-15 micrometers, or about 4-35 micrometers, or about 4-100 micrometers, or about 4-200 micrometers.

The device above, wherein from 1-90% of the flow in the device is within uniform flow channels, or from 1-75% of the flow in the device is within uniform flow channels, or from 1-50% of the flow in the device is within uniform flow channels, or from 1-25% of the flow in the device is within uniform flow channels.

The restriction channels and the uniform flow channels can be integral with the same chip or substrate. The restriction channels and the uniform flow channels may be in different chips or substrates. The restriction channels can be microfluidic channels.

The device above, further comprising means for analyzing the biological sample in the channels.

The device above, further comprising means for analyzing a proteomic composition, a lipidomic composition, a transcriptomic composition, or a carbohydrate composition of the biological sample in the channels.

The device above, further comprising means for measuring the level of the isolated fraction of the sample within the channels.

The device above, further comprising means for measuring the level of a biomarker in the isolated fraction of the sample within the channels.

The device above, wherein the plurality of obstructions comprise pillars integral with the channels.

The device above, wherein the plurality of obstructions comprise one or more of: a portion of a human or animal uveal meshwork, a portion of a human or animal corneoscleral meshwork, or a portion of a human or animal juxtacanalicular meshwork.

The plurality of obstructions may comprise glass beads, magnetic beads, gel particles, dextran particles, or polymer particles.

The biological sample can be composed of human or animal bodily fluid, blood, tissue, or cells. The biological sample comprises a carrier fluid.

The device above, wherein the biological sample comprises one or more reagents.

The device above, wherein the restriction channels further comprise binding moieties for binding a biomarker or biomolecule of the sample.

The biological sample may be from a subject undergoing a diagnosis or prognosis.

The device of above, further comprising a serpentine fluid mixing region in the restriction channels. The device above, wherein the restriction channels or continuous flow channels have a fluorinated coating.

This invention further contemplates methods for extracting extracellular matrix bodies from a biological sample, by

flowing the biological sample from the inlet end to the outlet end of a device of claim 1; and

reversing the direction of flow of a fluid toward the inlet end of the device.

A microfluidic system for isolating a fraction of a biological sample, the system comprising:

a microfluidic device comprising

one or more restriction channels having an inlet end and an outlet end, wherein the inlet end and outlet end are in fluid communication through the channel;

a plurality of spaced-apart obstructions lodged in the restriction channels for providing resistance to flow, wherein the spacing between obstructions decreases in the direction from the inlet end to the outlet end; and

an inlet reservoir for holding a fluid, wherein the fluid reservoir is in fluid communication with the inlet end of the restriction channels; and

one or more uniform flow channels having an inlet end and an outlet end, wherein the inlet end and outlet end are in fluid communication through the channel, wherein the inlet end is in fluid communication with the inlet reservoir;

a drive unit comprising a pressure source;

a source unit comprising a fluid source, wherein the pressure source is in fluid communication with the fluid source and the inlet reservoir of the microfluidic device;

a sensor unit comprising a sensor in fluid communication with the inlet reservoir for measuring the flow rate and pressure of the fluid at the inlet reservoir and sending the flow and pressure data to a processor; and

an on-chip analyzer unit comprising one or more means for analyzing the isolated fraction in the microfluidic device and sending the analysis data to a processor; and

a processor for receiving and displaying the flow rate, pressure and analysis.

A composition comprising a fraction of a biological sample extracted from a device of this disclosure. The composition may be used in the treatment of the human or animal body. The composition can be used in the diagnosis or prognosis of a subject.

A method for preparing a biological sample, the method comprising isolating extracellular matrix bodies from the biological sample. The extracellular matrix bodies may have a size from 0.5 to 5,000 micrometers, or from lto 1,000 micrometers, or from 1 to 200 micrometers, or from 4 to 100 micrometers. The isolating extracellular matrix bodies can be performed by ultrafiltration or centrifugation. The isolating extracellular matrix bodies can be performed by a device of this disclosure.

The method above, further comprising fixating the extracellular matrix bodies on a glass surface using 1-Ethyl-3-(3-dimethylaminopropyl) carbodiimide crosslinking.

A method for preparing a biological sample of extracellular matrix bodies fixating the extracellular matrix bodies on a glass surface using 1-Ethyl-3-(3-dimethylaminopropyl) carbodiimide crosslinking.

### BRIEF DESCRIPTION OF THE DRAWINGS

FIG. 1 shows a plan view of a microfluidic chip embodiment of this invention. In this format, a silicon wafer master 101 is printed with three microfluidic channel chip patterns 103. A silicon wafer 101 can be used as a substrate. Photoresist can be poured onto the substrate and exposed to UV light, which forms the pattern of the microfluidic chips 103. Together, the wafer and photoresist form a mold onto which PDMS can be poured. Once set, the PDMS can be peeled off the mold, giving three casts of microfluidic chips per wafer. These casts can be adhered to glass slides to form the final microfluidic chips.

FIG. 2 shows a plan view of a microfluidic chip insert in an embodiment of a device of this invention. The chip has two restriction channels 203, in this example each 2500 um wide and 25,000 um in length. The restriction channels 203 contain pillars of various diameters and spacing, shown by circles. The chip has a third uniform flow channel 205 having pillars of uniform size and spacing which do not significantly restrict the flow. The chip has an inlet reservoir 201 and an outlet reservoir 207, which also contain larger pillars. The dashed arrow shows the direction of flow from the inlet reservoir towards the outlet reservoir.

FIG. 3 shows a plan view corresponding to FIG. 2. FIG. 3 shows PDMS polymeric pillars 301 of various sizes represented by circles. The flow of biofluid through three channels is shown by dashed arrows.

FIG. 4 shows a plan view corresponding to the inlet reservoir of FIG. 2. FIG. 4 shows pillars 401 represented by circles. The flow of biofluid through three channels is shown by dashed arrows.

FIG. 5 shows a plan view corresponding to the inlet reservoir region of FIG. 2. FIG. 5 shows pillars 501 represented by circles. The flow of biofluid through three channels is shown by dashed arrows.

FIG. 6 shows a plan view corresponding to the channel region of FIG. 2. FIG. 6 shows pillars 601 represented by circles. The flow of biofluid through a channel is shown by a dashed arrow. The microfluidic channel device of this invention may have regions of different size and/or spacing of pillars or obstructions for creating turbulent or restricted flow.

FIG. 7 shows an expanded plan view corresponding to the channel region of FIG. 2. FIG. 7 shows pillars 701 represented by circles. The flow of biofluid through a channel is shown by a dashed arrow. This view shows a transition from 50 um gaps between pillars to 25 um gaps in a restriction channel.

FIG. 8 shows an expanded plan view corresponding to the channel region of FIG. 2. FIG. 8 shows pillars 801 represented by circles. The flow of biofluid through a channel is shown by a dashed arrow. This view shows a transition from larger to smaller gaps between pillars in a restriction channel.

FIG. 9 shows an expanded plan view corresponding to the channel region of FIG. 2. FIG. 9 shows pillars 901 represented by circles. The flow of biofluid through a channel is shown by a dashed arrow.

FIG. 10 shows an expanded plan view corresponding to the channel region of FIG. 2. FIG. 10 shows pillars 1001 represented by circles. The flow of biofluid through a channel is shown by a dashed arrow. This view shows channels having regions of blunt pillar obstructions 1001 which can create turbulent flow.

FIG. 11 shows an expanded plan view corresponding to the outlet reservoir 1107 of FIG. 2. FIG. 11 shows pillars 1101, 1103, and 1105 of various sizes. The flow of biofluid through a channel is shown by a dashed arrow. In this embodiment, the outer restriction channels each contain a barrier 1102 formed by very small and closely-spaced pillars.

FIG. 12 shows an expanded plan view corresponding to the inlet reservoir 1201 of FIG. 2. FIG. 12 shows pillars 1203 of various sizes. Outer restriction channel 1207 contains pillars of varying size and spacing. Uniform flow channel 1205 contains pillars of uniform size and spacing. The direction of flow of biofluid through an outer channel is shown by a dashed arrow.

FIG. 13 shows a plan view of a microfluidic chip in an embodiment of a device of this invention. Three microfluidic inserts are shown. The direction of flow of biofluid is shown by a dashed arrow.

FIG. 14 shows a perspective view of an embodiment of a microfluidic channel device of this invention having blunt pillar obstructions 1401 to flow. FIG. 14 is an expansion of FIG. 15. The direction of flow of biofluid is shown by dashed arrows.

FIG. 15 shows a perspective view of an embodiment of a microfluidic channel device of this invention. FIG. 15 shows a view corresponding to the channel region of FIG. 2. FIG. 15 shows blunt pillar obstructions 1501 of varying spacing in a restriction channel. In this embodiment, a restriction channel can have pillar obstructions 1501 organized in bands of varying spacing between the pillars. The direction of flow of biofluid is shown by a dashed arrow.

FIG. 16 shows an elevation side view of a microfluidic chip embodiment of this invention. The inlet reservoir 1605 is in fluid communication with a fluid line 1601 for introducing biofluid and/or other fluid into the reservoir. The fluid line 1601 passes through a probe 1602, probe adapter 1603, and hole 1604 defined in a glass cover slide. The biofluid passes through the inlet reservoir 1605 to reach the microfluidic channel 1606. The direction of flow of biofluid is shown by a dashed arrow.

FIG. 17 shows an expanded plan view corresponding to the inlet region of FIG. 2, and the position of a probe 1602 of FIG. 16. The direction of flow of biofluid is shown by a dashed arrow.

FIG. 18 shows an elevation side view of a microfluidic chip 1614 embodiment of this invention. The inlet reservoir is in fluid communication with a fluid line 1601 for introducing biofluid into the reservoir. The fluid line 1601 passes through a probe 1602, probe adapter 1603, and hole 1604 defined in a glass cover slide 1613. The biofluid passes through the inlet reservoir to reach the microfluidic channel 1606 and flow to the outlet reservoir 1607. A probe adjuster 1612 can be provided to adjust the height of the probe 1602 to create a good seal with the probe adapter 1603 and hole 1604. The direction of flow of biofluid is shown by a dashed arrow.

FIG. 19 shows an expanded plan view corresponding to the channel region of FIG. 2. FIG. 19 shows pillars 1701 represented by circles. For this embodiment, some representative lengths of regions of pillar bands in the outer channel are shown in micrometers.

FIG. 20 shows a micrograph of an expanded plan view corresponding to the channel region of FIG. 2. FIG. 20 shows pillars as dots. For this embodiment, some representative lengths of regions of pillar bands in the outer channel are shown in micrometers. The direction of flow of biofluid is shown by a dashed arrow.

FIG. 21 shows a plan view of an embodiment of a microfluidic device corresponding to FIG. 2. Biofluid can be introduced with a delivery probe 2201 to the inlet region reservoir 2202. The direction of flow of biofluid to the outlet reservoir region 2203 is shown by a dashed arrow. An expansion view for this embodiment shows some representative lengths of regions of pillar bands in the outer channel in micrometers. For this embodiment, dotted lines in the expansion view show possible tortuous paths of biofluid amongst the obstructions.

FIG. 22 shows an embodiment of a microfluidic system of this invention having a processor, a fluid drive unit, a fluid source unit, a sensor unit, an on-chip unit, and an off-chip unit.

FIG. 23 shows that aqueous humor from a patient with primary open angle glaucoma increased the pressure in the microfluidic device. FIG. 23 shows the relative amount of pressure (mm Hg) change within an artificial trabecular meshwork formed by pillars in a microfluidic channel when infused with human aqueous humor obtained from a patient with severe primary open angle glaucoma. The fluid flow rate was held constant at 2 μl per minute, and the baseline system pressure was measured using an external pressure sensor. The human aqueous humor sample was injected at timepoint denoted by an arrow and the letter “a.” The pressure steadily rises to a maximum of about 41 mm Hg at 27 minutes. FIG. 23 shows that aqueous humor from patients diagnosed with POAG glaucoma increased the pressure in the device.

FIG. 24 (top) shows a confocal photomicrograph of a microfluidic chip after capturing EMB from human aqueous humor from a patient with primary open angle glaucoma. Protein of the EMB was labeled with a fluorescent marker, carboxyfluorescein succinimidyl ester (CFSE, marked with arrows). The circles are pillars in a restriction channel. FIG. 24 (lower) shows EMB isolated in the microfluid channels around pillars.

FIG. 25 illustrates isolation of extracellular matrix bodies from a biofluid using size exclusion filters. Bovine vitreous humor was filtered with a 5 μm cellulose acetate syringe filter, followed by a 1 μm syringe-tip filter, and subsequently a 0.45 um syringe-tip filter, and then a 0.22 μm filter. Each fraction was characterized using wide-field microscopy. FIG. 25 shows unenriched bovine vitreous humor 4301 was aspirated into a 1 mL syringe with a 22 g needle 4305 and extruded through a 5 μm syringe-tip filter 4309. The filtrate was collected and filtered through a 1 μm syringe-tip filter 4311. The filtrate was collected and filtered through a 0.45 μm syringe-tip filter 4313. The filtrate was collected and extruded through a 0.22 μm syringe-tip filter 4315. Biofluid fractions were collected after each filtration step for optical microscopy. The microscopy images showed a reduction in larger bodies as the filtrate was serially passed through smaller filter sizes.

FIG. 26 shows representative transmission electron microscopy (TEM) images for isolation of EMB by size exclusion filters. FIG. 26a and FIG. 26b show the presence of extracellular matrix bodies present in the native biofluid of the bovine vitreous humor. To isolate and recover extracellular matrix bodies from a complex biofluid, serial syringe-based filtration was performed with cellulose filter from 5 μm to 0.22 μm pore sizes. Extracellular matrix bodies were stained with alcian blue stain. FIG. 26c shows bovine vitreous fractions isolated by serial filtration through a 5 μm syringe-tip filter. FIG. 26d shows bovine vitreous fractions isolated by serial filtration through a 1 μm syringe-tip filter. FIG. 26e shows bovine vitreous fractions isolated by serial filtration through a 0.45 μm syringe-tip filter. FIG. 26f shows bovine vitreous fractions isolated by serial filtration through a 0.22 μm syringe-tip filter. The images show a relative reduction in larger ECM bodies as the filtrate was serially passed through smaller filter sizes.

FIG. 27 illustrates isolation of extracellular matrix bodies from a biofluid using centrifugation. Four pellets 9705, 9713, 9721, and 9729 were obtained by serial centrifugation. Bovine vitreous was re-suspended 9701 and was placed in 1 ml tubes and centrifuged (Sorvall Legend RT) at 350 g at 4° C. for 10 minutes to form Pellet 1 9705. A 50 μl aliquot of the supernatant was saved for analysis and labeled Supernatant 1 9709, the remaining supernatant was transferred to a new tube and centrifuged (Eppendorf, 5417R series, F45-30-11 Eppendorf rotor) at 2000 g at 4° C. for 10 minutes to form Pellet 2 9713. A 50 μl aliquot of the supernatant was saved for analysis and labeled Supernatant 2 9717, the remaining supernatant was transferred to a new tube. The supernatant was then centrifuged at 10,000 g at 4° C. for 10 minutes to form Pellet 3 9721. A 50 μl aliquot of the supernatant was saved for analysis and labeled Supernatant 3 9725, and the remaining supernatant was transferred to a new tube. The supernatant was then centrifuged at 20,000 g at 4° C. for 10 minutes to give Pellet 4 9729. A 50 μl aliquot of the supernatant was saved for analysis and labeled Supernatant 4, and the remaining supernatant was transferred to a new tube.

FIG. 28 shows representative transmission electron microscopy (TEM) images for isolation of EMB by serial centrifugation. FIG. 28a shows extracellular matrix bodies present in the bovine vitreous humor. In FIG. 28b, a representative TEM photomicrograph of sample collected from the pellet after centrifugation at 450 g showed extracellular matrix bodies present in the pellet fraction. Likewise, in FIG. 28c, a representative TEM photomicrograph of sample collected from the pellet after centrifugation at 2,000 g showed extracellular matrix bodies present in the pellet fraction. In FIG. 28d, a representative TEM photomicrograph of sample collected from the pellet after centrifugation at 10,000 g showed extracellular matrix bodies present in the pellet fraction. In FIG. 28e, a representative TEM photomicrograph of sample collected from the pellet after centrifugation at 20,000 g showed extracellular matrix bodies present in the pellet fraction.

FIG. 29 shows the dose-response behavior of the compound bivalirudin TFA on intraocular pressure (IOP) in bovine vitreous humor. Differential pressure is measured with a microfluidic device of this invention.

FIG. 30 shows the dose-response behavior of the compound colistin sulfate on intraocular pressure (IOP) in bovine vitreous humor. Differential pressure is measured with a microfluidic device of this invention.

FIG. 31 shows the dose-response behavior of the compound polymyxin B sulfate on intraocular pressure (IOP) in bovine vitreous humor glaucoma model. Differential pressure is measured with a microfluidic device of this invention.

FIG. 32 shows isolation and extraction of extracellular matrix bodies in a restriction channel of a microfluidic device of this disclosure. FIG. 32a and FIG. 32a show representative photomicrographs of a microfluidic chip perfused with a biofluid of bovine vitreous humor. The letter “p” marks a pillar in the channel. After perfusion, extracellular matrix bodies were isolated between and around the pillars. FIG. 32c and FIG. 32d show representative photomicrographs of the channels after extracting extracellular matrix bodies. Extracellular matrix bodies were dislodged from the chip, which showed substantially fewer bodies after extraction.

FIG. 33 shows proteomic analysis off-chip of isolated and extracted bovine extracellular matrix bodies by LC/MS. Biomarkers for extracellular matrix bodies were detected.

FIG. 34 shows isolation of extracellular matrix bodies in a restriction channel of a microfluidic device of this disclosure and their subsequent extraction. Image scale bars are 50 μm. FIG. 34a shows representative widefield photomicrographs of a microfluidic device perfused with bovine vitreous humor suspended in phosphate-buffered saline, pH 7.0 and counterstained for hyaluronic acid with alcian blue (grey signal, brightfield) FIG. 34a shows the signal from extracellular matrix bodies (arrows) trapped between the pillars (p) of the device. The chip was perfused with the biofluid for at least 60 minutes. After perfusion, the aggregates were isolated between pillars, observed in a mass-like formation. Material smaller than the extracellular matrix bodies material had exited via the outlet port. FIG. 34b shows extraction of extracellular matrix bodies from a restriction channel of a microfluidic device. The device was perfused with a mild detergent, 0.1% sodium dodecyl sulfate, SDS, and reversed flow direction from outlet to inlet. FIG. 34b exhibited substantially fewer bodies present in the channel after elution, and showed that the bodies were extracted. FIG. 34c shows a higher power image of extracellular matrix bodies (arrows) trapped between the pillars (p) after perfusion. FIG. 34d shows extraction with detergent and reverse flow, again showing substantially fewer bodies in the channel after extraction.

FIG. 35 shows on-chip immunohistochemical staining of extracellular matrix bodies in a device channel of this disclosure. The microfluidic device was infused with a fluid containing homogenized bovine vitreous suspended in a biofluid. After perfusion of the fluid into the device, the fluid flowed through the inlet and exited via the outlet. The larger extracellular matrix bodies (arrows) were trapped between the pillars (marked Lp). The chip was perfused with a blocking solution to prevent non-specific antibody binding before antibody staining. Next, protein fibronectin, a known extracellular matrix component and integrin-binding protein, was labeled by infusing anti-fibronectin primary antibody, incubating the sample for 2 hours, and washing. Then, goat anti-rabbit FITC secondary antibody was incubated for 1 hour and washed. The microfluidic chip was then imaged under wide-field fluorescence and brightfield microscopy. FIG. 35 shows a representative wide-field-fluorescent photomicrograph. This image shows extracellular matrix bodies in a microfluidic channel. The distance between large pillars (Lp) was about 100 μm. The punctate signal within the bodies represents fibronectin staining (anti-fibronectin Ab, goat anti-rabbit secondary antibody with Alexa 488, FITC, white signal).

FIG. 36 shows on-chip immunohistochemical staining of extracellular matrix bodies in a device channel of this disclosure. FIG. 36a shows a representative photomicrograph brightfield image of the stained extracellular matrix bodies in a channel (arrows). Image scale bar was 20 μm. Control images had no fluorescent signal, which showed that the signal in FIG. 36a was specific for fibronectin. FIG. 36b again shows stained extracellular matrix bodies in a channel (arrow). Image scale bar was 50 μm. Again, control images had no fluorescent signal, which showed that the signal in FIG. 36b was specific for fibronectin.

FIG. 37 shows on-chip immunohistochemical staining of extracellular matrix bodies in a device channel of this disclosure. FIG. 37 shows a representative photomicrograph of on-chip immunohistochemical staining of perlecan protein, a component of the extracellular matrix of cartilage, a known cancer biomarker, in a biofluid containing extracellular matrix bodies. The microfluidic chip was infused with a fluid containing homogenized bovine vitreous suspended in a biofluid. After perfusion of the biofluid into the device, the sample flowed through the inlet and exited via the outlet. The larger extracellular matrix bodies (arrows) were trapped between pillars (marked “p”). The chip was perfused with a blocking solution to prevent non-specific antibody binding before antibody staining. Perlecan was labeled by infusing anti-perlecan primary antibody, incubating the sample for 2 hours, and washing. Then, goat anti-rabbit TRITC secondary antibody was incubated for 1 hour and washed. The microfluidic chip was then imaged under wide-field fluorescence and brightfield microscopy. FIG. 37 shows extracellular matrix bodies in a microfluidic channel between pillars (p). The punctate signal represents perlecan staining (white signal). Control images had no fluorescent signal, which showed that the signal in FIG. 37 was specific for perlecan.

FIG. 38 shows on-chip immunohistochemical staining of extracellular matrix bodies in a device channel of this disclosure. FIG. 38a shows a representative photomicrograph brightfield image of on-chip immunohistochemical staining of perlecan. Control images had no fluorescent signal, which showed that the signal in FIG. 38a was specific for perlecan. Image scale bar was 10 μm. FIG. 38b shows a representative photomicrograph brightfield image of on-chip immunohistochemical staining of perlecan. Control images had no fluorescent signal, which showed that the signal in FIG. 38b was specific for perlecan. Image scale bar was 10 μm. FIG. 38 also shows that alcian blue, a marker for hyaluronic acid, can be used as a stain for EMB.

FIG. 39 shows extracellular matrix bodies were visualized on a glass surface using 1-Ethyl-3-(3-dimethylaminopropyl) carbodiimide crosslinking and staining of collagen using picrosirius red dye. FIG. 39 shows a signal (dark stain) for extracellular matrix bodies, which showed that EDC crosslinking retained the extracellular matrix bodies on the surface.

FIG. 40 shows extracellular matrix bodies were visualized on a glass surface using 1-Ethyl-3-(3-dimethylaminopropyl) carbodiimide crosslinking and staining of collagen using picrosirius red dye. FIG. 40 shows a signal (dark stain) for collagen strands within extracellular matrix bodies, which showed that EDC crosslinking retained the extracellular matrix bodies on the surface. FIG. 40 also shows that the stain picrosirius red can be used to stain EMB.

FIG. 41 shows off-chip analysis extracellular matrix bodies were visualized on a glass surface using 1-Ethyl-3-(3-dimethylaminopropyl) carbodiimide crosslinking and staining of DNA using Hoechst dye. FIG. 41 shows signal for DNA (Hoechst dye, DAPI filter, white signal, DNA) in extracellular matrix bodies.

FIG. 42 shows on-chip isolation and detection of extracellular matrix bodies. FIG. 42 shows a representative photomicrograph of a microfluidic chip perfused with bovine vitreous humor suspended in the phosphate-buffered saline, pH 7.0, and counterstained for hyaluronic acid with alcian blue, dark stain, brightfield. FIG. 42 shows signal from extracellular matrix bodies trapped near a large pillar (circle) of the device. The chip was perfused with the biofluid for at least 60 minutes. Extracellular matrix bodies were observed in a cluster-like formation. Image scale bar was 50 μm.

FIG. 43 shows on-chip isolation, detection and analysis of extracellular matrix bodies in a microfluidic device. FIG. 43 shows a representative low-power fluorescent photomicrograph of a channel after perfusion of bovine vitreous humor extracellular matrix bodies that were counterstained for protein with carboxyfluorescein succinimidyl ester (CFSE). The microfluidic chip was infused with a fluid containing homogenized bovine vitreous. After perfusion of the fluid into the device, the fluid flowed through the inlet and exited via the outlet. The larger extracellular matrix bodies were trapped near pillars (marked “p”). Image scale bar was 25 μm.

FIG. 44 shows on-chip isolation, detection and analysis of extracellular matrix bodies in a microfluidic device. FIG. 44 shows a representative photomicrograph of a microfluidic chip perfused with bovine vitreous humor suspended in the phosphate-buffered saline, pH 7.0, and counterstained for collagen with picrosirius red, dark stain, brightfield. FIG. 44a shows signal from extracellular matrix bodies between pillars (marked “p’) of the device. The chip was perfused with the biofluid for at least 60 minutes. FIG. 44b shows the same image with a fluorescent filter, and shows that collagen was detected with picrosirius red (light grey signal). Image scale bar was 50 μm. FIG. 44c and FIG. 44d show similar images at a higher power. Image scale bar was 10 μm.

FIG. 45 shows frequency size distribution of human extracellular matrix bodies present in human aqueous humor biofluids from healthy and pre-disease states, glaucoma suspect and pre-glaucoma. Aqueous humor was obtained from 8 patients, healthy sample or pre-glaucoma diagnosis, having intraocular pressures ranging from 9 to 25 mmHg. The human samples were not processed by centrifugation or other means. The size of extracellular matrix bodies was determined by crosslinking sample to a glass slide using a carbodiimide EDC fixative, staining with uranyl acetate, and imaging with wide-field microscopy. The size was quantified using an automated program (ImageJ) in all eight samples. The size (area) of extracellular matrix bodies ranged from about 1.67 μm² to about 67×10³ μm². FIG. 45 shows the count of extracellular matrix bodies in the 0-200 μm² range.

FIG. 46 shows frequency size distribution of human extracellular matrix bodies present in human aqueous humor biofluids from healthy and pre-disease states, glaucoma suspect and pre-glaucoma. Aqueous humor was obtained from 8 patients, healthy sample or pre-glaucoma diagnosis, having intraocular pressures ranging from 9 to 25 mmHg. The human samples were not processed by centrifugation or other means. The size of extracellular matrix bodies was determined by crosslinking sample to a glass slide using a carbodiimide EDC fixative, staining with uranyl acetate, and imaging with wide-field microscopy. The size was quantified using an automated program (ImageJ) in all eight samples. The size (area) of extracellular matrix bodies ranged from about 1.67 μm² to about 67×10³ μm². FIG. 46 shows the count of extracellular matrix bodies in the 201-1000 μm² range.

FIG. 47 shows frequency size distribution of human extracellular matrix bodies present in human aqueous humor biofluids from healthy and pre-disease states, glaucoma suspect and pre-glaucoma. Aqueous humor was obtained from 8 patients, healthy sample or pre-glaucoma diagnosis, having intraocular pressures ranging from 9 to 25 mmHg. The human samples were not processed by centrifugation or other means. The size of extracellular matrix bodies was determined by crosslinking sample to a glass slide using a carbodiimide EDC fixative, staining with uranyl acetate, and imaging with wide-field microscopy. The size was quantified using an automated program (ImageJ) in all eight samples. The size (area) of extracellular matrix bodies ranged from about 1.67 μm² to about 67×10³ μm². FIG. 47 shows the count of extracellular matrix bodies in the 1001-67,000 μm² range.

FIG. 48 shows size distribution of bovine vitreous extracellular matrix bodies isolated and extracted from a microfluidic device of this invention. Extracellular matrix bodies in bovine vitreous humor biofluid after isolation and extraction from a microfluidic device of this invention. The chip was perfused with the biofluid for at least 60 minutes. After 60 minutes of perfusion, the ECM bodies were isolated near restriction channel pillars. Next, the chip was treated with a detergent (0.1% sodium dodecyl sulfate, SDS), and the sample extracted from the chip via reverse flow, which allowed the bodies to flow out of the inlet port. Fractions of the eluate were collected at 10-minute intervals for a total of 80 minutes. Sample was mounted on a glass slide and stained with alcian blue, and imaged with wide-field microscopy. The size was quantified using an automated program (ImageJ). The size (area) of extracellular matrix bodies was up to about 16×10³ μm². FIG. 48 shows the count of extracellular matrix bodies in each eluate fraction, which increased over time. This experiment showed that a microfluidic device of this invention can be used to isolate and extract extracellular matrix bodies of various sizes.

FIG. 49 shows for off-chip analysis of extracellular matrix bodies can be done with retained with 1-ethyl-3-(3-dimethylaminopropyl) carbodiimide (EDC) crosslinking. FIG. 49a shows a representative TEM image of extracellular matrix bodies from native bovine vitreous humor obtained with EDC crosslinking. Extracellular matrix bodies were observed with EDC fixation. FIG. 49b shows a similar image taken without EDC crosslinking. Extracellular matrix bodies were not observed without EDC fixation.

FIG. 50 shows isolation of extracellular matrix bodies from a human plasma sample from a patient with early-stage pancreatic ductal adenocarcinoma (PDAC) as compared to a healthy control. FIG. 50a shows the representative wide-field-fluorescent photomicrograph image for the PDAC sample. FIG. 50a shows extracellular matrix bodies lodged in a microfluidic channel. FIG. 50a further shows the larger extracellular matrix bodies (arrows) were lodged between pillars (marked “p”). The width between pillars was about 100 μm. The punctate signal from the lodged extracellular matrix bodies represents fibronectin staining (anti-fibronectin Ab, goat anti-rabbit secondary Ab with Alexa 488, FITC, white signal). The staining showed an abundant signal and punctate staining within the EMB (FIG. 50a, arrowheads). FIG. 50b shows a similarly-obtained fluorescent photomicrograph of an age-matched healthy control human plasma sample. FIG. 50b shows a markedly reduced amount of fibronectin signal (FIG. 50b, grey signal, arrow). The healthy control signal was far smaller than for the disease signal when processed under identical conditions. Image scale bars were 20 μm.

### DETAILED DESCRIPTION OF THE INVENTION

This invention discloses devices and systems for isolating particles from a biological sample applicable to various diseases and conditions by isolating and using biological bodies, materials and/or molecules. The devices and systems for isolating particles from a biological sample can be used to increase the level of sample isolates relevant to the biology of interest. Devices and methods of this invention can be used for isolating significant components from a biological sample for use in diagnosis and prognosis of disease.

The devices of this invention for isolating particles from a biological sample can be used with various kinds of biological materials and samples, including bodily fluids, blood, tissues, cells and tumors. In certain embodiments, methods of this disclosure can isolate relevant fractions of a biological material corresponding to a disease state.

This disclosure provides devices that can be used for isolating a significant fraction of a biological sample, with analysis of its components related to diagnosis and prognosis of disease. In some embodiments, a significant amount of biological material, and correspondingly improved signal level, can be obtained.

Aspects of this invention include isolating and preserving the composition and properties of extracellular matrix bodies (EMB) from a biological fluid or material. By preserving the composition and properties of extracellular matrix bodies (EMB) isolated or extracted from a biological sample, fluid or material, the EMB can be used for diagnosis or indication of disease, or for monitoring chemical or biological processes or changes of the sample material.

In some aspects, this invention provides enhanced ability to detect or characterize extracellular matrix bodies, which may more readily reflect a disease state.

In additional aspects, devices and methods of this disclosure can provide increased signal for pertinent biomarkers which relate a specific biological fraction to a disease state. Biomarkers of this disclosure can be associated with a disease and provide a diagnostic tool.

A device of this disclosure can provide improved measurement of flow and/or pressure in a fluid containing biological components by maintaining a continuous and substantial flow in the device. In some embodiments, when a substantial flow is maintained, a sensor can precisely measure differential pressure and flow. Devices and system disclosed herein can provide improved measures of flow and pressure in a fluid containing biological components by arrangements of channels that maintain continuous and substantial flow. In some embodiments, a device of this disclosure may have one or more channels that do not substantially restrict the flow of a fluid so that a continuous and substantial flow is maintained in the system.

Devices disclosed herein can provide improved measures of flow and pressure in a fluid containing biological components by using one or more uniform flow channels that maintain continuous and substantial flow, in addition to restriction channels.

In certain embodiments, a device of this invention will have from 1-90% of the flow from uniform flow channels, or from 1-75% of the flow from uniform flow channels, or from 1-50% of the flow from uniform flow channels, or from 1-25% of the flow from uniform flow channels.

In certain embodiments, a device of this invention will have at least 25% of the flow from uniform flow channels, or at least 50% of the flow from uniform flow channels, or at least 75% of the flow from uniform flow channels, or at least 90% of the flow from uniform flow channels.

In some embodiments, extracellular matrix bodies of diameter size from about 0.5 to about 5,000 micrometers or larger can be lodged in a microfluidic channel, or can cause a blockage in the channel, so that pressure is increased.

In additional embodiments, extracellular matrix bodies of diameter size from about 1 to about 200 micrometers or larger can be lodged in a microfluidic channel, or can cause a blockage in the channel, so that pressure is increased.

In further aspects, this disclosure provides devices and methods for isolating, detecting, and/or analyzing ultrastructural components of a fluid containing biological material or molecules. In certain embodiments, ultrastructural components may be associated with disease.

Embodiments of this invention can be used to isolate, extract, and utilize extracellular matrix bodies (EMB) that are a source of multiple and specific biomarkers.

This invention includes devices for isolating, detecting and analyzing compositions of extracellular matrix bodies, biological particles and complexes for various uses.

In certain aspects, extracellular matrix bodies can operate as biomarkers through their morphological features. In further aspects, extracellular matrix bodies can operate by containing isolated biochemical markers that may be presented in disease pathways.

Aspects of this invention can further provide a diagnostics system including devices for detecting and measuring biomarkers via disease-associated extracellular matrix bodies (EMB) and/or biological particles or complexes.

In further embodiments, this disclosure describes methods and devices for preparing and analyzing a sample of a biological material.

Extracellular matrix bodies (EMB) can be complexes, and may be composed of proteins, lipids, carbohydrates, nucleic acid molecules, bioparticles, small vesicles such as extracellular vesicles or exosomes, and combinations thereof.

Samples of a biological material can include bodily fluids, tissues, and cells.

Examples of samples of bodily fluids include any bodily fluid, including whole blood, blood plasma, blood components, CSF, urine, seminal fluid, synovial fluid, pleural fluid, vaginal fluid, gastric fluid, pericardial fluid, peritoneal fluid, amniotic fluid, saliva, nasal fluid, otic fluid, breast milk, and any other bodily fluid, and combinations thereof.

In further aspects, a microfluidic device and system of this invention can be used for isolating and extracting bioparticles from a sample.

A microfluidic device and system of this invention can be used for purifying or separating extracellular matrix bodies or complexes greater than about 0.5 micrometer in diameter up to particles about 5,000 micrometer in diameter, or greater than about 2 micrometer in diameter up to particles about 700 micrometer in diameter.

In further aspects, a microfluidic device and system of this invention can be used for measuring the relative viscosity and flow properties of biological and clinical fluids.

In certain aspects, a microfluidic device and system of this invention can be used for isolating and extracting bioparticles from a sample that are associated with a disease.

In some aspects, a microfluidic device and system of this invention can be used for measuring intraocular pressure in ocular fluids.

### Devices and Systems

This invention provides microfluidic devices and systems for measuring pressure and/or flow in a fluid.

This invention improves measurement of flow and pressure in a fluid by providing a continuous and substantial rate and volume of flow, so that differential pressure and/or flow can be measured precisely.

In further aspects, a microfluidic device and system of this invention can be used for detecting, isolating and extracting bioparticles or other components of biological materials or fluids.

A microfluidic device and system of this invention can be used for measuring the relative viscosity and flow properties of biological and clinical fluids.

A microfluidic device and system of this invention may comprise a microfluidic chip that can be held in a substrate.

In some aspects, a microfluidic device of this invention can have channels with obstructions. An obstruction can affect and/or restrict the flow of a fluid in the channel.

In some aspects, a microfluidic device of this invention can have channels with bands of obstructions. A band of obstructions can traverse the width of a channel so that the band of obstructions will affect fluid flow and flux along the channel.

In some embodiments, the spacing between obstructions may be constant in a band. The spacing between obstructions in a channel may be used to control the size of fenestrations in the channel for fluid flow.

In additional embodiments, a microfluidic device of this invention can have a channel with a plurality of sequential bands of obstructions. In certain embodiments, the spacing between obstructions in a band can decrease in the plurality of bands along the length of the channel in one direction. Consequently, the spacing between obstructions in a band can increase in the plurality of bands along the length of the channel in the opposite direction.

For example, a microfluidic device of this invention can have a restriction channel in which a plurality of bands in sequence in one direction have spacings between obstructions of 1,000 micrometers, which is adjacent to a band with spacings of 500 micrometers, which is adjacent to a band with spacings of 200 micrometers, which is adjacent to a band with spacings of 100 micrometers, which is adjacent to a band with spacings of 50 micrometers, which is adjacent to a band with spacings of 25 micrometers, which is adjacent to a band with spacings of 10 micrometers, which is adjacent to a band with spacings of 4 micrometers.

In further aspects, a microfluidic device of this invention can have channels with bands of obstructions, where the spacing between obstructions in one band is at a minimum. A band having the spacing between obstructions at a minimum can be a barrier band.

In additional aspects, a microfluidic device of this invention can have a restriction channel in which a barrier band has a spacing between obstructions of at least 1 micrometer, or at least 2, or at least 4, or at least 5, or at least 10, or at least 50, or at least 100, or at least 200 micrometers.

In certain aspects, a microfluidic device of this invention can be a chip of from 7 to 25 micrometer in height.

In operation, a microfluidic device and system of this invention can be used for isolating, extracting, and/or purifying bioparticles. In certain embodiments, a restriction channel may have bands of decreasing spacing between obstructions, where one band is a barrier band. Methods of this invention can use such an arrangement of bands to isolate larger particles from smaller particles and fluid by flowing the fluid containing the particles from an inlet in the direction of decreasing spacing towards an outlet of the channel. In these methods, larger particles can be isolated and retained along the channel, while smaller particles and fluid exit the channel at the outlet. The larger particles can be extracted from the channel at the inlet by reversing the direction of flow of an extracting fluid to extract the larger particles from the inlet.

In operation, a microfluidic device and system of this invention can be used for isolating, extracting, and/or purifying bioparticles extracted from the channel at the outlet.

In some embodiments, larger particles can be extracted from the channel at the outlet with the addition of a detergent to break up the larger particles.

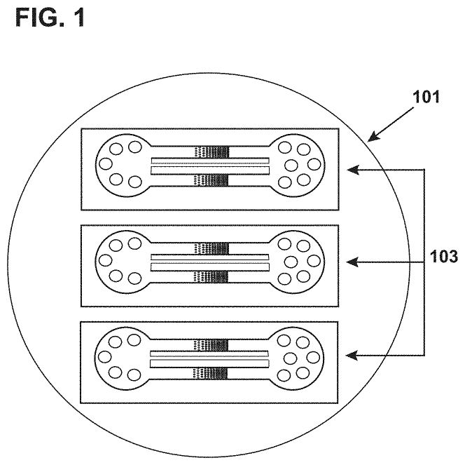

FIG. 1 shows a plan view of a microfluidic chip embodiment of this invention. In this format, a silicon wafer master 101 is printed with three microfluidic channel chip patterns 103. A silicon wafer 101 can be used as a substrate. Photoresist can be poured onto the substrate and exposed to UV light, which forms the pattern of the microfluidic chips 103. Together, the wafer and photoresist form a mold onto which PDMS can be poured. Once set, the PDMS can be peeled off the mold, giving three casts of microfluidic chips per wafer. These casts can be adhered to glass slides to form the final microfluidic chips.

A microfluidic chip of this invention can have a channel for restricted flow of a fluid, and an inlet and an outlet for fluid flow. A pump may be used to apply head pressure of a fluid at the inlet. In some embodiments, a reduced or vacuum pressure can be used at the outlet to adjust flow.

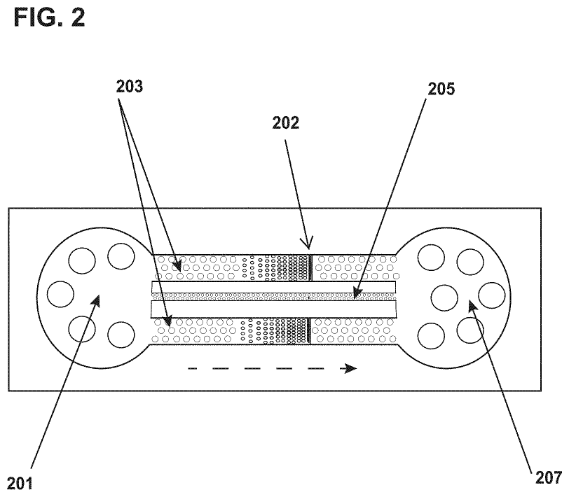

FIG. 2 shows a plan view of a microfluidic chip insert in an embodiment of a device of this invention. The chip has two restriction channels 203, in this example each 2,500 um wide and 25,000 um in length. The restriction channels 203 contain pillars of various diameters and spacing, shown by circles. The chip has a third uniform flow channel 205 having pillars of uniform size and spacing which do not significantly restrict the flow. The chip has an inlet reservoir 201 and an outlet reservoir 207, which also contain larger pillars. The dashed arrow shows the direction of flow from the inlet reservoir towards the outlet reservoir. A restriction channel can have a point of maximal restriction to flow, which is a barrier 202 to flow. The barrier 202 can restrict flow and alter pressure in the channel and system, so that differential pressure and/or flow can be related to composition of the fluid.

A microfluidic chip of this invention can have one or more channels for restricted flow of a fluid, and one or more uniform or continuous flow channels. In some embodiments, the uniform flow channel does not present a restriction to fluid flow in the channel. The uniform continuous flow channel may contain blunt obstructions for creating turbulent flow and/or a tortuous path for flowing fluid.

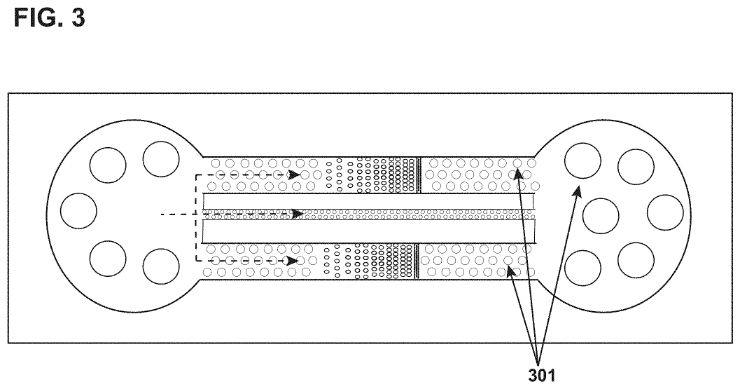

FIG. 3 shows a plan view corresponding to FIG. 2. FIG. 3 shows PDMS polymeric pillars 301 of various sizes represented by circles. The flow of biofluid through three channels is shown by dashed arrows.

In certain embodiments, blunt or non-blunt obstructions may be provided in a restriction fluid channel to create a tortuous or vortex pattern of flow in certain regions. Obstructions in a channel can be formed as pillars of circular or other shapes.

In certain embodiments, the obstructions of a restriction channel may provide a Reynolds number of greater than 500, or greater than 1000, or greater than 10,000, or greater.

In additional embodiments, a continuous uniform flow channel may be located in between various restriction channels. In certain embodiments, uniform flow channels and restriction channels can have any order of arrangement and be used in any number.

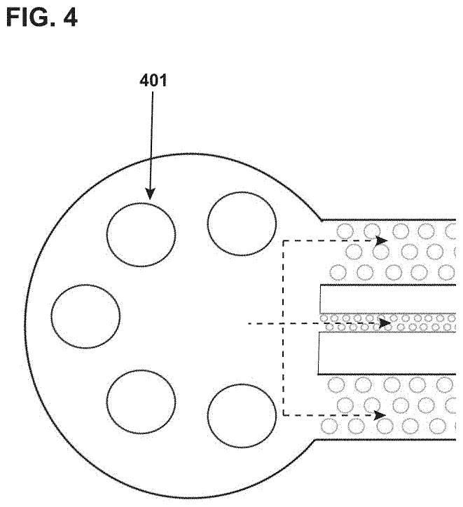

FIG. 4 shows a plan view corresponding to the inlet reservoir of FIG. 2. FIG. 4 shows pillars 401 represented by circles. The flow of biofluid through three channels is shown by dashed arrows.

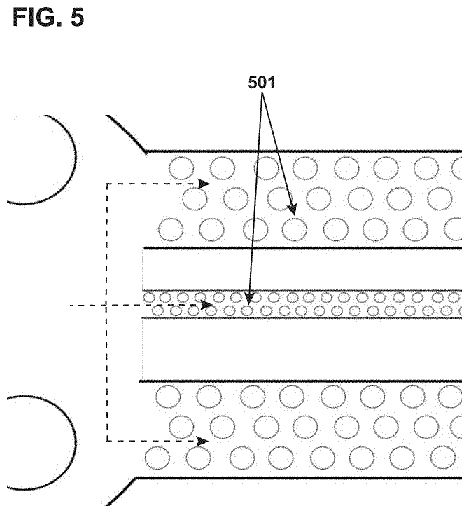

FIG. 5 shows a plan view corresponding to the inlet reservoir region of FIG. 2. FIG. 5 shows pillars 501 represented by circles. The flow of biofluid through three channels is shown by dashed arrows.

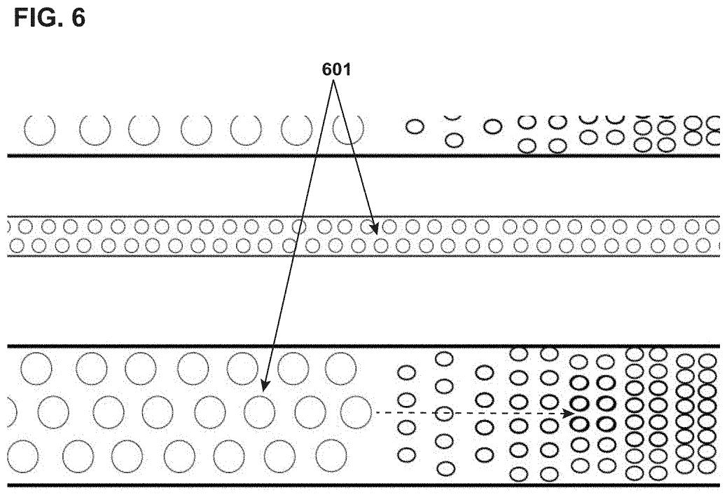

FIG. 6 shows a plan view corresponding to the channel region of FIG. 2. FIG. 6 shows pillars 601 represented by circles. The flow of biofluid through a channel is shown by a dashed arrow. The microfluidic channel device of this invention has regions of different spacing and/or size of pillars or obstructions creating turbulent or restricted flow.

In certain embodiments, a microfluidic channel device of this disclosure may have regions simulating an ocular trabecular mesh.

A device of this invention may include a meshwork composition which contains extracellular matrix bodies or complexes. Extracellular matrix bodies or complexes for use in a meshwork composition may be extracted or purified from glaucoma ocular humor. The ocular humor may be from animal or clinical sources.

In further embodiments, a microfluidic chip of this invention can have a one or more channels for restricted flow of a fluid and one or more uniform flow channels. The uniform flow channels may contain blunt obstructions for creating turbulent flow and/or a tortuous path for flowing fluid.

In further embodiments, a microfluidic chip of this invention can have a 1-20 channels for restricted flow of a fluid and 1-10 uniform flow channels, arranged in any order on a substrate. The uniform flow channels may be distributed in any manner with respect to the restricted flow channels.

In certain embodiments, uniform flow channels may alternate in co-linear or parallel positions with respect to restricted flow channels. In additional embodiments, uniform flow channels may be above or below restricted flow channels. In some embodiments, uniform flow channels may be arranged in a separate substrate from a chip that contains restriction flow channels.

In further embodiments, the uniform flow channels may provide fluid communication from an inlet reservoir to an outlet reservoir. In certain embodiments, a uniform flow channel may provide fluid communication from an outlet reservoir to the source of the fluid entering an inlet reservoir.

In certain embodiments, the total cross sectional area of uniform flow channels may be greater than, or less than the total cross sectional area of restriction flow channels in a microfluidic device of this invention. In various embodiments, uniform flow channels may not contain obstructions and may not have tortuous fluid flow. In such embodiments, uniform flow channels can have laminar or turbulent fluid flow.

A microfluidic chip of this invention can have one or more restriction channels for restricted flow of a fluid. The restricted flow may be due to various arrangements of blunt or non-blunt obstructions or pillars in the channel. In some embodiments, the pillars may present a shape to the flowing fluid, such as circular, spherical, triangular, square, polygonal, diamond, fin-shaped, and combinations thereof.

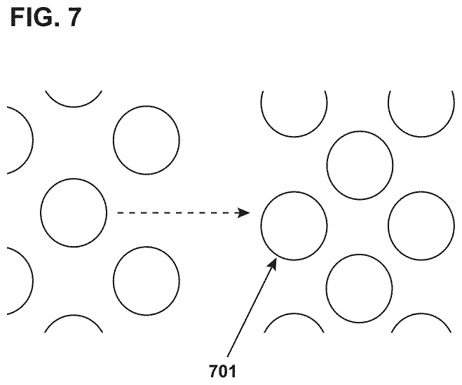

FIG. 7 shows an expanded plan view corresponding to the channel region of FIG. 2. FIG. 7 shows pillars 701 represented by circles. The flow of biofluid through a channel is shown by a dashed arrow. This view shows a transition from 50 um gaps between pillars to 25 um gaps in a restriction channel.

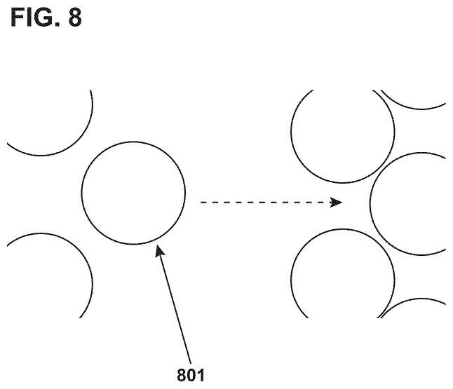

FIG. 8 shows an expanded plan view corresponding to the channel region of FIG. 2. FIG. 8 shows pillars 801 represented by circles. The flow of biofluid through a channel is shown by a dashed arrow. This view shows a transition from larger to smaller gaps between pillars in a restriction channel.

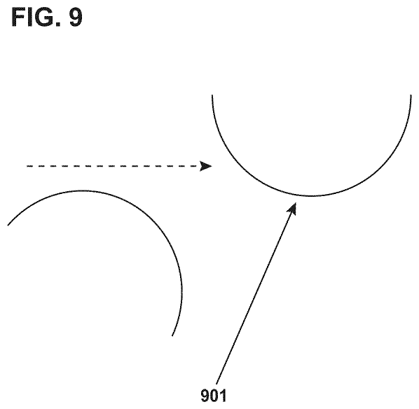

FIG. 9 shows an expanded plan view corresponding to the channel region of FIG. 2. FIG. 9 shows pillars 901 represented by circles. The flow of biofluid through a channel is shown by a dashed arrow.

In further embodiments, restricted flow in a channel may be due to various arrangements of blunt or non-blunt obstructions or pillars in the channel, where the size and spacing of obstructions changes with distance along the channel.

In certain embodiments, the size and/or spacing of blunt or non-blunt obstructions or pillars in a restriction channel may change with distance along the channel. The size and/or spacing of blunt or non-blunt obstructions may reduce with distance along the channel. At some position in a restriction channel, the size and/or spacing of blunt or non-blunt obstructions may be reduced to a level which provides a maximal restriction or barrier to flow.

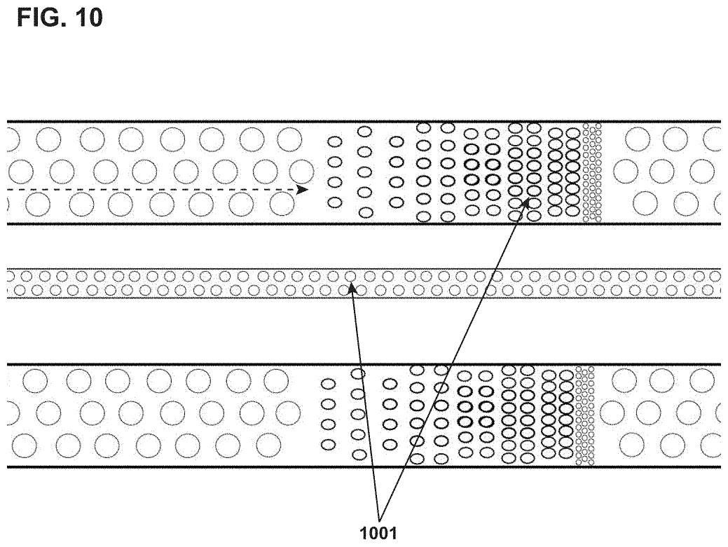

FIG. 10 shows an expanded plan view corresponding to the channel region of FIG. 2. FIG. 10 shows pillars 1001 represented by circles. The flow of biofluid through a channel is shown by a dashed arrow. This view shows channels having regions of blunt pillar obstructions 1001 which can create turbulent flow.

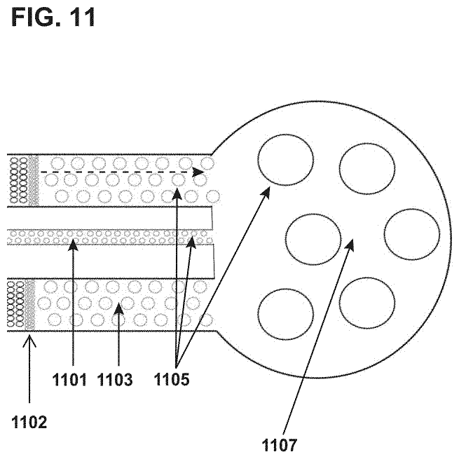

FIG. 11 shows an expanded plan view corresponding to the outlet reservoir 1107 of FIG. 2. FIG. 11 shows pillars 1101, 1103, and 1105 of various sizes. The flow of biofluid through a channel is shown by a dashed arrow. In this embodiment, the outer restriction channels each contain a barrier 1102 formed by very small and closely-spaced pillars.

In further embodiments, various arrangement of blunt or non-blunt obstructions or pillars in a restriction channel can be used to restrict flow to any level. A wide range of spacings and/or patterns of blunt and/or non-blunt obstructions can be used in a restriction channel. A fluid may have a tortuous path in a restriction flow channel. The spacing of obstructions in a restriction channel and/or the tortuosity of the fluid path can increase with distance along the channel in the direction of flow.

Fluid effluents from the channels of a microfluidic chip of this invention can be collected in an outlet reservoir at the outlet end of the channels. The inflow or insertion of fluid to the channels of a microfluidic chip of this invention can be achieved with a reservoir at the inlet end of the channels.

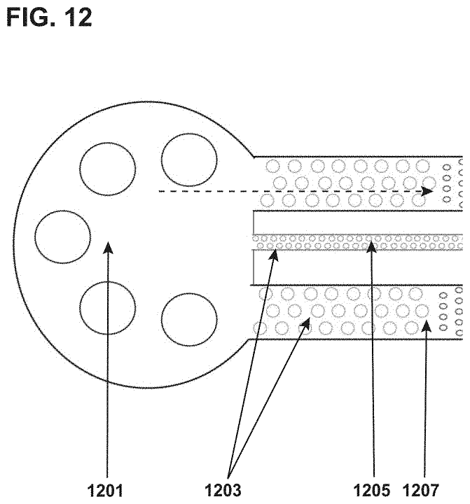

FIG. 12 shows an expanded plan view corresponding to the inlet reservoir 1201 of FIG. 2. FIG. 12 shows pillars 1203 of various sizes. Outer restriction channel 1207 contains pillars of varying size and spacing. Uniform flow channel 1205 contains pillars of uniform size and spacing. The direction of flow of biofluid through an outer channel is shown by a dashed arrow.

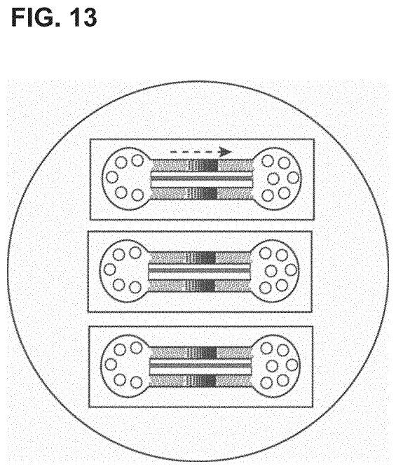

FIG. 13 shows a plan view of a microfluidic chip in an embodiment of a device of this invention. Three microfluidic inserts are shown. The direction of flow of biofluid is shown by a dashed arrow.

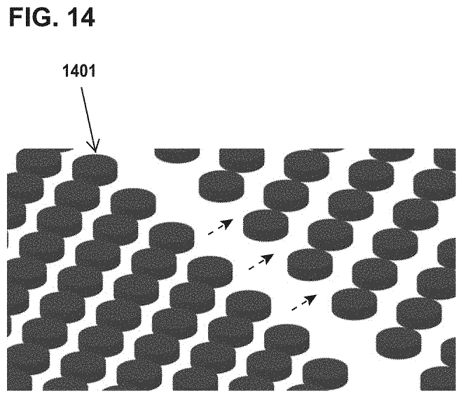

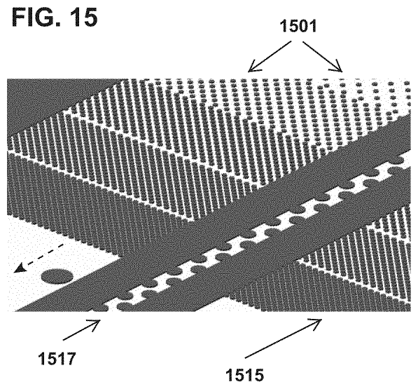

FIG. 14 shows a perspective view of an embodiment of a microfluidic channel device of this invention having blunt pillar obstructions 1401 to flow. FIG. 14 is an expansion of FIG. 15. The direction of flow of biofluid is shown by dashed arrows.

FIG. 15 shows a perspective view of an embodiment of a microfluidic channel device of this invention. FIG. 15 shows a view corresponding to the channel region of FIG. 2. FIG. 15 shows pillar obstructions 1501 of varying spacing in a restriction channel. In this embodiment, a restriction channel can have pillar obstructions 1501 organized in bands of varying spacing between the pillars. The direction of flow of biofluid is shown by a dashed arrow. A continuous uniform flow channel 1517 can be arranged separate from a restriction channel 1515.

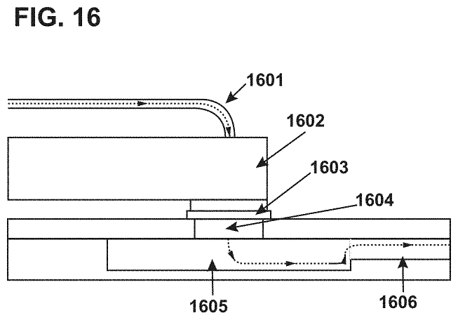

FIG. 16 shows an elevation side view of a microfluidic chip embodiment of this invention. The inlet reservoir 1605 is in fluid communication with a fluid line 1601 for introducing biofluid and/or other fluid into the reservoir. The fluid line 1601 passes through a probe 1602, probe adapter 1603, and hole 1604 defined in a glass cover slide. The biofluid passes through the inlet reservoir 1605 to reach the microfluidic channel 1606. The direction of flow of biofluid is shown by a dashed arrow.

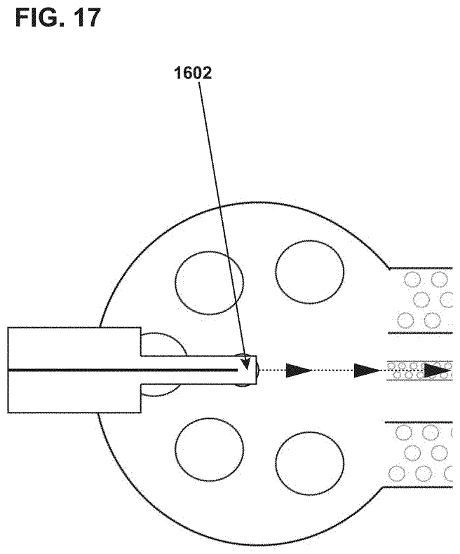

FIG. 17 shows an expanded plan view corresponding to the inlet region of FIG. 2, and the position of a probe 1602 of FIG. 16. The direction of flow of biofluid is shown by a dashed arrow.

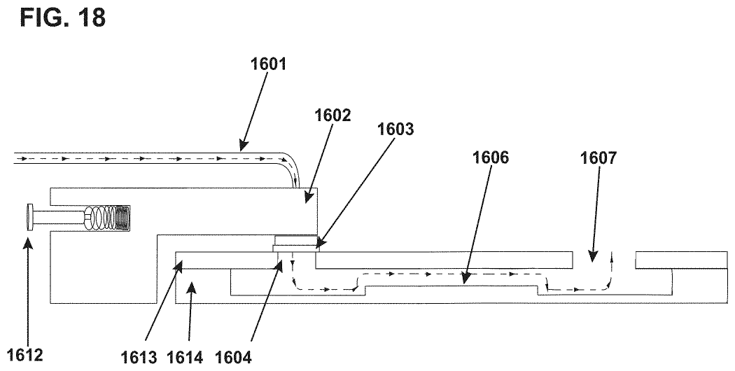

FIG. 18 shows an elevation side view of a microfluidic chip 1614 embodiment of this invention. The inlet reservoir is in fluid communication with a fluid line 1601 for introducing biofluid into the reservoir. The fluid line 1601 passes through a probe 1602, probe adapter 1603, and hole 1604 defined in a glass cover slide 1613. The biofluid passes through the inlet reservoir to reach the microfluidic channel 1606 and flow to the outlet reservoir 1607. A probe adjuster 1612 can be provided to adjust the height of the probe 1602 to create a good seal with the probe adapter 1603 and hole 1604. The direction of flow of biofluid is shown by a dashed arrow.

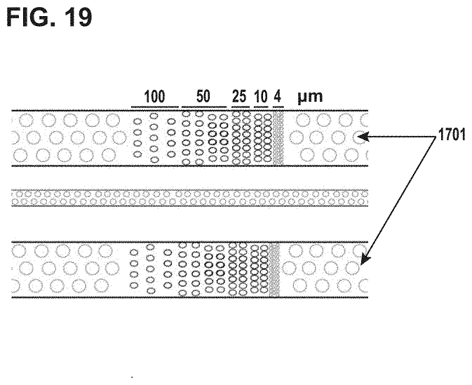

FIG. 19 shows an expanded plan view corresponding to the channel region of FIG. 2. FIG. 19 shows pillars 1701 represented by circles. For this embodiment, some representative lengths of regions of pillar bands in a channel are shown in micrometers.

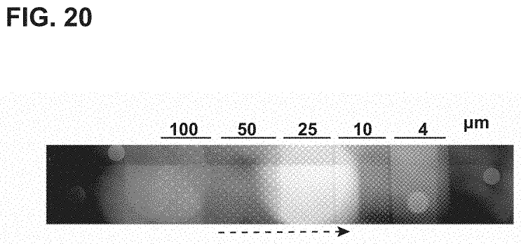

FIG. 20 shows a micrograph of an expanded plan view corresponding to the channel region of FIG. 2. FIG. 20 shows pillars as dots. For this embodiment, some representative spacings of pillars in a band in a channel are shown in micrometers. The direction of flow of biofluid is shown by a dashed arrow.

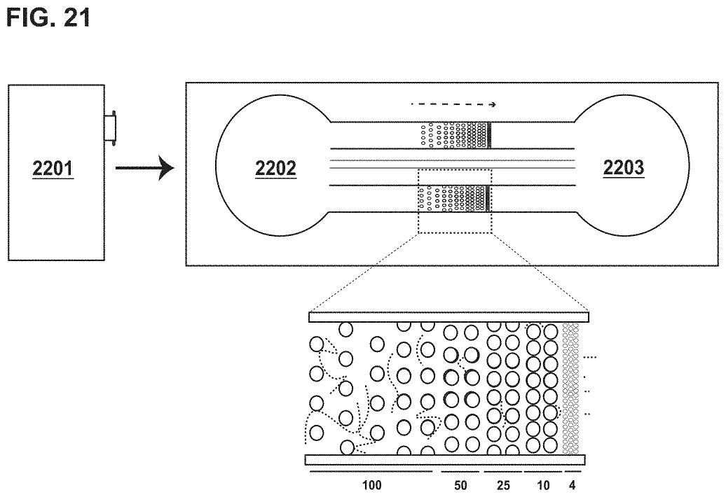

FIG. 21 shows a plan view of an embodiment of a microfluidic device corresponding to FIG. 2. FIG. 21 shows biofluid can be introduced with a delivery probe 2201 to the inlet region reservoir 2202. The direction of flow of biofluid to the outlet reservoir region 2203 is shown by a dashed arrow. An expansion view for this embodiment shows some representative spacing of pillars in bands in a channel in micrometers. For this embodiment, dotted lines in the expansion view show possible tortuous paths of biofluid amongst the obstructions.

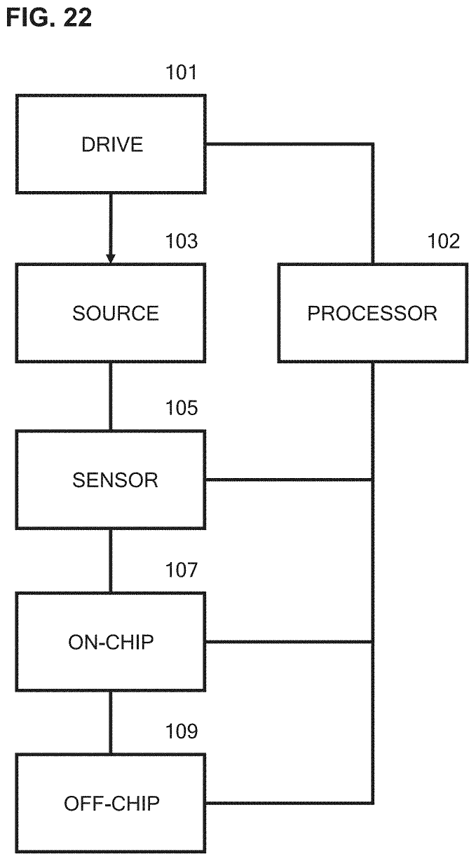

FIG. 22 shows an embodiment of a microfluidic system of this invention. A processor 102 can send control signals and/or receive signals from a fluid drive unit 101, which provides a drive fluid, such as a compressed gas, to a fluid source unit 103. The fluid source unit 103 can contain a fluid, biofluid, carrier, and/or reagents of interest. The fluid, biofluid, carrier, and/or reagents of interest can flow to a sensor unit 105, which can monitor flow rate and/or pressure of the fluid. The fluid, biofluid, carrier, and/or reagents of interest can flow to an on-chip unit 107, which may include a microfluidic device of this invention. The fluid, biofluid, carrier, and/or reagents of interest can enter the inlet reservoir of a microfluidic chip of this invention in the on-chip unit 107. The fluid, biofluid, carrier, and/or reagents of interest can reach the outlet reservoir of a microfluidic chip of this invention in the on-chip unit 107 and flow to an off-chip unit 109. The processor 102 can receive data from the sensor unit 105, and record the flow and/or pressure. The on-chip unit 107 can include analytical tools such as irradiation and light detectors for spectrometry. The off-chip unit 109 can include various analytical tools such as microscopy tools, imagers, and analyzers, chromatography analyzers, mass spectrometry analyzers, and/or magnetic resonance analyzers. The processor 102 can send control signals and/or receive data from the on-chip unit 107 and off-chip unit 109.

In some aspects, a fluid composition in a system or device of this invention can be analyzed by various techniques. For example, a fluid composition can be analyzed by an imaging technique.

Examples of imaging techniques include electron microscopy, stereoscopic microscopy, wide-field microscopy, polarizing microscopy, phase contrast microscopy, multiphoton microscopy, differential interference contrast microscopy, fluorescence microscopy, laser scanning confocal microscopy, multiphoton excitation microscopy, ray microscopy, and ultrasonic microscopy.

Examples of imaging techniques include positron emission tomography, computerized tomography, and magnetic resonance imaging.

Examples of assay techniques include colorimetric assay, chemiluminescence assay, spectrophotometry, immunofluorescence assay, and light scattering.

In some embodiments, this invention can provide a device for measuring pressure and flow rate of a fluid composition. In certain embodiments, a device can have a meshwork composition lodged in the channel for providing resistance to flow. The meshwork composition may have any one or more of a uveal meshwork, a corneoscleral meshwork, and a juxtacanalicular meshwork. Such meshworks can be simulated with obstructions in a restriction channel, for example, or provided from extraction of ocular humor, bodily fluid, or clinical samples.

Extracellular matrix bodies or complexes for use in a meshwork composition may be composed of various biomolecules or complexed particles, and may have diameters ranging from about 0.5 to about 5,000, or from 0.5 to 1,000, or from 1 to 200, or from 1 to 100, or from 1 to 50, or from 1 to 25, or from 1 to 10, or from 1 to 5 micrometers.

Extracellular matrix bodies or complexes that can be isolated in a device of this invention may have diameters ranging from about 0.5 to about 5,000, or from 0.5 to 1,000, or from 2 to 700, or from 1 to 200, or from 1 to 100, or from 1 to 50, or from 1 to 25, or from 1 to 10, or from 1 to 5 micrometers.

In some embodiments, a channel may contain obstructions such as glass beads, micro beads, magnetic beads, gel particles, dextran particles, or polymer particles. Obstructions may also be composed of glass fibers, polymeric fibers, inorganic fibers, organic fibers, or metal fibers.

In additional embodiments, a device or channel of this invention may include binding agents, affinity detectors, or immuno-agents attached to the elements of the device which are in fluid communication with the sample fluid. A device may have agents for internal capture and/or detection of biological molecules from a sample. In certain embodiments, a device can have agents for internal capture and/or detection of biomarkers of a sample fluid.

In certain embodiments, a uveal meshwork or restriction channel may have fenestrations of about 25 micrometers. A corneoscleral meshwork or restriction channel may have fenestrations of about 2-15 micrometers. A juxtacanalicular meshwork or restriction channel may have fenestrations of about 1 to 4 micrometers or less.

A device may further include a fluid reservoir for holding a fluid composition, so that the fluid reservoir is in fluid communication with the inlet of a channel for introducing the fluid composition into the inlet of the channel.

A device of this disclosure can have a drive or pressure source for applying pressure to a drive fluid composition. The drive fluid can enter a fluid reservoir for driving the fluid composition into the inlet of a microfluidic channel.

A device of this invention can have a sensor unit in fluid communication with the fluid composition for measuring the flow rate and pressure of the fluid composition at the inlet of the channel and transmitting the flow rate and pressure to a processor.

Signals and data from units of the system device can be received by a processor. The processor can display the flow rate and pressure. Memory or media can store instructions or files, such as a machine-readable storage medium. A machine-readable storage medium can be non-transitory.

A processor of this disclosure can be a general purpose or special purpose computer. A processor can execute instructions stored in a machine readable storage device or medium. A processor can include an integrated circuit chip, a microprocessor, a controller, a digital signal processor, any of which can be used to receive and/or transmit data and execute stored instructions. A processor can also perform calculations and transform data, and/or store data in a memory, media or a file. A processor may receive and execute instructions which may include performing one or more steps of a method of this invention. A device of this invention can include one or more non-transitory machine-readable storage media, one or more processors, one or more memory devices, and/or one or more user interfaces. A processor may have an integral display for displaying data or transformed data.

In some aspects, a system of this disclosure may have a device having microfluidic channels. One or more channels can be arranged in a microfluidic chip.

A system of this disclosure can include an on-chip unit having one or more detectors for analyzing the fluid composition within the channels or at the inlet or exiting the outlet of the channel. Detectors can also be arranged to detect the fluid composition within the channel.

A system of this disclosure can include an off-chip unit having one or more detectors for analyzing a fluid composition extracted from microfluidic channels.

In certain embodiments, extracellular matrix bodies or complexes for use in a meshwork composition in a system or device of this disclosure may include a fixative, a stabilizing component, or a cross linking component which can transform the structure to a stable, uniform composition.

Examples of stabilizing components include fixatives as described herein, cross linking compounds as described herein, organic solvents, polypeptides, and pharmaceutically-acceptable organic salts.

Extracellular matrix bodies or complexes that are cross linked can be reversibly cross linked, or non-reversibly cross linked.

In some embodiments, a device of this invention may contain extracellular matrix bodies or complexes as a meshwork composition that can be used for identifying or screening active agents. A meshwork composition may include a drug delivery excipient.

In additional embodiments, a device of this invention may be used for measuring the quantity or level of extracellular matrix bodies or complexes in a test sample. Measuring the quantity or level of extracellular matrix bodies or complexes in a test sample can provide a diagnostic marker level for the test sample. A device of this invention can be used to identify glaucoma or pre-glaucoma in a subject.

In further embodiments, a device of this invention may be used for measuring a pressure which can be related to a quantity or level of extracellular matrix bodies or complexes in a test sample. A pressure value in a channel can be related directly to a quantity or level of extracellular matrix bodies or complexes in a test sample.

In certain embodiments, a device of this invention may be used for measuring an assay value which can be related to a quantity or level of extracellular matrix bodies or complexes in a test sample. An assay value of a composition in a channel can be related directly to a quantity or level of extracellular matrix bodies or complexes in a test sample.

Example of an assay include a colorimetric assay, a chemiluminescence assay, a spectrophotometry assay, an immunoassay, or a light scattering assay.

Means for analyzing a sample in a microfluidic device include analytical tools such as irradiation sources and light detectors for spectrometry and spectroscopy, as well as immuno-labeling and detection, as further shown in the examples herein.

Means for analyzing a sample in a microfluidic device include imaging tools such as irradiation sources and microscopy, as further shown in the examples herein.

### Extracted Compositions and Methods

In some embodiments, a composition may comprise a fraction of a biological sample extracted from a microfluidic device.

In certain embodiments, a composition extracted from a microfluidic device may be used in the treatment of the human or animal body.

In additional embodiments, a composition extracted from a microfluidic device may be used in the diagnosis or prognosis of a subject.

A composition of extracellular matrix bodies, isolated and/or extracted, can be combined with a pharmaceutical carrier and one or more pharmaceutical excipients.

The morphology of extracellular matrix bodies, isolated and/or extracted, may be modified by the isolation and/or extraction processes.

The morphology of extracellular matrix bodies, isolated and/or extracted, may be chemically-modified.

In some embodiments, a composition of extracellular matrix bodies can be isolated and/or extracted for use in the treatment of the human or animal body.

In further embodiments, a composition may comprise a sample from which extracellular matrix bodies have been removed by the isolation and/or extraction processes for use in the treatment of the human or animal body. In certain embodiments, at least 25%, or at least 50%, or at least 75%, or at least 90% of the extracellular matrix bodies of a sample have been removed by the isolation and/or extraction processes for use in the treatment of the human or animal body.

In some aspects, extracting a composition from a microfluidic device may be a method for preparing a biological sample for use in the diagnosis or prognosis of a subject.

In certain embodiments, methods for isolating extracellular matrix bodies can be performed by ultrafiltration or centrifugation, or by a microfluidic device of this disclosure.

Embodiments of this invention further include fixation of extracellular matrix bodies on a glass surface using 1-Ethyl-3-(3-dimethylaminopropyl) carbodiimide crosslinking.

All publications including patents, patent application publications, and non-patent publications referred to in this description are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The terms “a,” “an,” “the,” and similar terms describing the invention, and in the claims, are to be construed to include both the singular and the plural.

### EXAMPLES

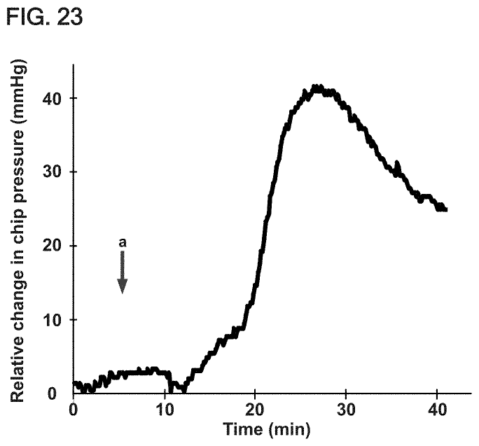

Example 1. Pressure and flow measurements in microfluidic device using disease-associated biofluid. Isolation of extracellular matrix bodies in a microfluidic device. FIG. 23 shows that aqueous humor from a patient with primary open angle glaucoma increased the pressure in the microfluidic device. FIG. 23 shows the relative amount of pressure (mm Hg) change within a microfluidic model trabecular meshwork when infused with human aqueous humor obtained from a patient with severe primary open angle glaucoma. The microfluidic channel flow rate was held constant at 2 μl per minute, and the baseline system pressure was measured using an external pressure sensor. The human aqueous humor sample was injected at timepoint denoted by an arrow and the letter “a.” The pressure steadily rises to a maximum of about 41 mm Hg at 27 minutes. FIG. 23 shows that aqueous humor from patients diagnosed with POAG glaucoma increased the pressure in the device.

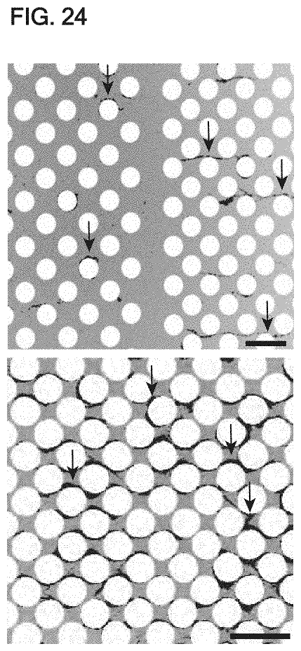

Example 2. Isolation of disease-associated extracellular matrix bodies in microfluidic device. FIG. 24 (top) shows a confocal photomicrograph of a microfluidic chip after isolating EMB from human aqueous humor from a patient with primary open angle glaucoma, at the end of the experiment shown in FIG. 23. FIG. 24 (top) shows protein content in the aqueous humor was labeled with a fluorescent marker, carboxyfluorescein succinimidyl ester (arrows). The circles are pillars in the restriction channel. FIG. 24 (lower) shows EMB isolated in the microfluid channels trapped between pillars (arrows).

Example 3. Isolation of extracellular matrix bodies from a biofluid using size exclusion filters. This example shows that EMB can be isolated by size exclusion. EMB can be isolated by size exclusion and distinguished from smaller particles, for example free extracellular vesicles or other small vesicles.

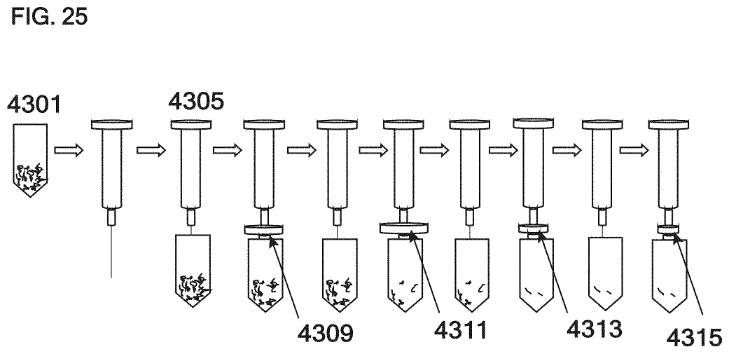

FIG. 25 illustrates isolation of extracellular matrix bodies from a biofluid using size exclusion filters. Bovine vitreous humor was filtered with a 5 μm cellulose acetate syringe filter, followed by a 1 μm syringe-tip filter, and subsequently a 0.45 um syringe-tip filter, and then a 0.22 μm filter. Each fraction was characterized using wide-field microscopy.

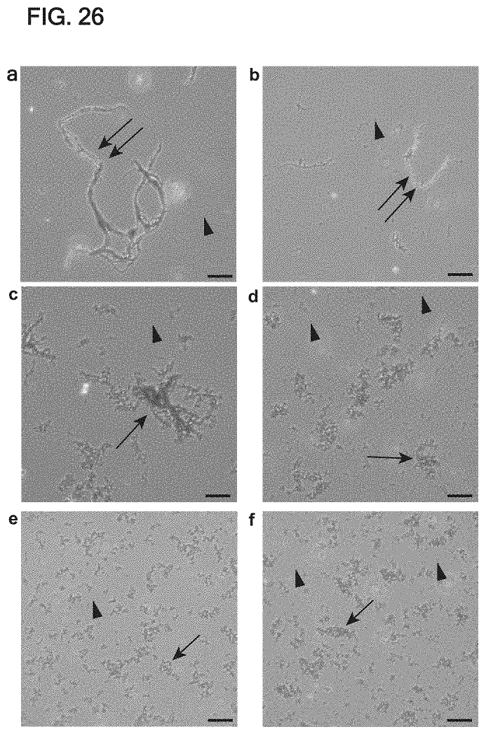

FIG. 26 shows representative wide field microscopy images for isolation of EMB by size exclusion filters. FIG. 26a and FIG. 26b show the presence of extracellular matrix bodies present in the native biofluid of the bovine vitreous humor. To isolate and recover extracellular matrix bodies from a complex biofluid, serial syringe-based filtration was performed with cellulose filter from 5 μm to 0.22 μm pore sizes. Extracellular matrix bodies were fixed to a glass slide with EDC and stained with alcian blue stain. FIG. 26c shows bovine vitreous fractions isolated by serial filtration through a 5 μm syringe-tip filter. FIG. 26d shows bovine vitreous fractions isolated by serial filtration through a 1 μm syringe-tip filter. FIG. 26e shows bovine vitreous fractions isolated by serial filtration through a 0.45 μm syringe-tip filter. FIG. 26f shows bovine vitreous fractions isolated by serial filtration through a 0.22 μm syringe-tip filter. The images show a relative reduction in larger ECM bodies as the filtrate was serially passed through smaller filter sizes.

This example shows that EMB can be isolated by size exclusion. Analysis of isolated EMB showed that it was composed of DNA, RNA, and protein, as well as hyaluronic acid, or collagen, which are components of the extracellular matrix.

The vitreous humor is a highly hydrated tissue having a water content of between 98-99.7%, which is essentially composed of extracellular matrix. A major component of the extracellular matrix is the protein collagen. Collagen proteins are modified with carbohydrates, and once released from a cell, are assembled into collagen fibrils. Extracellular matrix bodies can be attached to the fibrils, among other places in the extracellular matrix.

Bovine eyes were dissected to remove orbital fat and extraocular muscles attached to the globe. The globe was rinsed with 5 ml of ice-cold Tris Buffered Saline (TBS) containing 50 mM Tris-HCl, 150 mM NaCl (pH 8.0) for 1 minute at 4° C. Vitreous was dissected by making an sclerotomy incision 4 mm or 8 mm posterior to the limbus using a 16 g needle and then making a circumferential sagittal incision with scissors to separate the globe into an anterior and posterior cup. Scissors were used to cut and remove the formed vitreous and to sever adhesions between vitreous and ocular structures. Tissue samples were rinsed with TBS (pH 8.0) for 1 min at 4° C. Vitreous specimens were collected in 15 mL centrifugation tubes and homogenized using an immersion blender. Aliquots of homogenized bovine vitreous humor (BVH) were transferred to 1 mL centrifugation tubes. The bovine vitreous bodies were resuspended in TBS buffer for further studies and frozen at −80° C. until use.

An aliquot of homogenized bovine vitreous humor diluted to 1 mL with buffered saline was loaded into a 1 mL syringe using a 22-gauge needle. The needle was replaced with a 5 μm cellulose acetate syringe filter, and the bovine vitreous fluid was extruded through the filter by applying uniform downward pressure. The filtrate was collected into a new 1 mL tube, and an aliquot of 80 μL of the filtrate was saved for imaging. The filtrate collected from the 5 μm filtration was then loaded into a 1 μm syringe-tip filter following the same procedure described above, and an aliquot was saved for imaging. Next, the filtrate from the 1 μm filter was extruded through a 0.45 um syringe-tip filter following the same procedure, and an aliquot was saved for imaging. Finally, the filtrate from the 0.45 μm filter was extruded through a 0.22 μm filter. The filtrates recovered from each filtration step.

Wide-field microscopy was used to visualize components of each fraction. Each sample was imaged using by placing the biofluid on a glass slide, crosslinking the sample with EDC, and staining the hyaluronic acid-containing materials with Alcian blue. For staining of the samples, each filtrate was incubated 1:1 v/v with 1% Alcian blue (Sigma 1% Alcian Blue in 3% Acetic Acid pH 2.5 B8483) at room temperature for 30 minutes. After the incubation, 40 μL of the stained filtrate was placed onto a glass slide and covered with a coverslip, and imaged using bright-field microscopy. Color bright field images were captured on an inverted phase contrast microscope (Ziess Axiovert 200) equipped with an Axiocam 105 color camera (Zeiss), and images were processed with Zen software (Zeiss, version 4.3).

Example 4. Isolation of extracellular matrix bodies from a biofluid using serial centrifugation. This example shows that EMB can be isolated by centrifugation. EMB can be isolated by centrifugation and distinguished from smaller particles, for example free exosomes or other small vesicles. To obtain vitreous specimens free of cells, the vitreous was first cleared with a series of low-speed centrifugations.

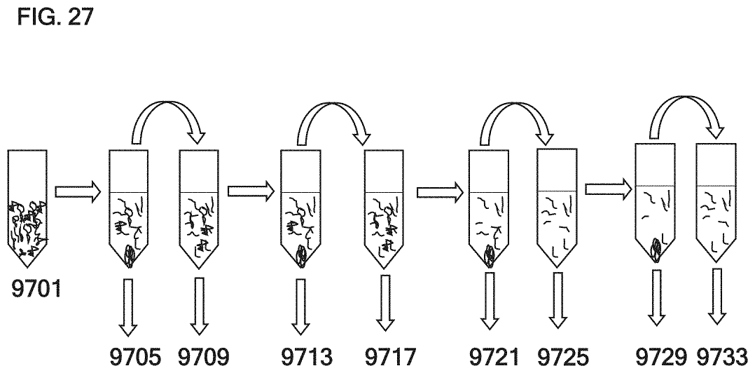

FIG. 27 illustrates methods for isolation of extracellular matrix bodies from a biofluid using centrifugation. Four pellets 9705, 9713, 9721, and 9729 were obtained by serial centrifugation. Bovine vitreous was re-suspended 9701 and was placed in 1 ml tubes and centrifuged (Sorvall Legend RT) at 350 g at 4° C. for 10 minutes to form Pellet 1 9705. A 50 μl aliquot of the supernatant was saved for analysis and labeled Supernatant 1 9709, the remaining supernatant was transferred to a new tube and centrifuged (Eppendorf, 5417R series, F45-30-11 Eppendorf rotor) at 2000 g at 4° C. for 10 minutes to form Pellet 2 9713. A 50 μl aliquot of the supernatant was saved for analysis and labeled Supernatant 2 9717, the remaining supernatant was transferred to a new tube. The supernatant was then centrifuged at 10,000 g at 4° C. for 10 minutes to form Pellet 3 9721. A 50 μl aliquot of the supernatant was saved for analysis and labeled Supernatant 3 9725, and the remaining supernatant was transferred to a new tube. The supernatant was then centrifuged at 20,000 g at 4° C. for 10 minutes to give Pellet 4 9729. A 50 μl aliquot of the supernatant was saved for analysis and labeled Supernatant 4, and the remaining supernatant was transferred to a new tube.

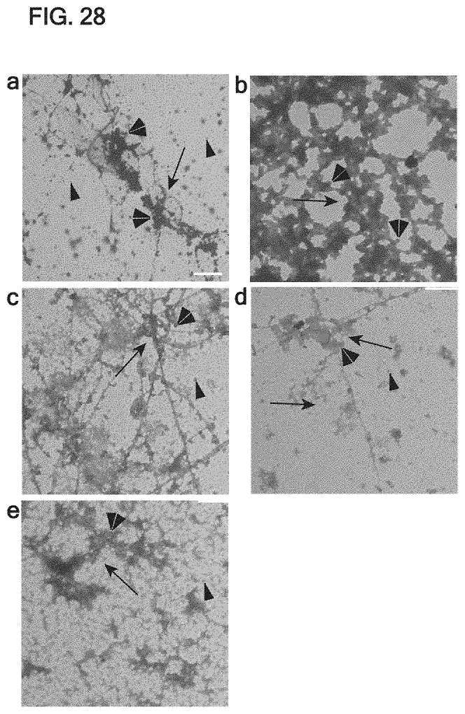

FIG. 28 shows representative transmission electron microscopy (TEM) images for isolation of EMB by serial centrifugation. FIG. 28a shows extracellular matrix bodies present in the bovine vitreous humor. In FIG. 28b, a representative TEM photomicrograph of sample collected from the pellet after centrifugation at 450 g showed extracellular matrix bodies present in the pellet fraction. Likewise, in FIG. 28c, a representative TEM photomicrograph of sample collected from the pellet after centrifugation at 2,000 g showed extracellular matrix bodies present in the pellet fraction. In FIG. 28d, a representative TEM photomicrograph of sample collected from the pellet after centrifugation at 10,000 g showed extracellular matrix bodies present in the pellet fraction. In FIG. 27e, a representative TEM photomicrograph of sample collected from the pellet after centrifugation at 20,000 g showed extracellular matrix bodies present in the pellet fraction.

Example 5. Dose-response in microfluidic device for detecting activity of agents in reducing intraocular pressure in a glaucoma model. The dose-response behavior of bivalirudin TFA on intraocular pressure (IOP) was determined for its use as active agent in treating glaucoma. Bivalirudin TFA exhibited an EC50 of 1.2 nM for treatment of bovine vitreous humor.

The compound bivalirudin TFA was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ethanol or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

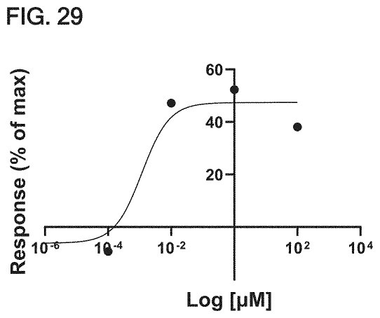

FIG. 29 shows the dose dependent response curve for the treatment of bovine vitreous humor with the compound bivalirudin TFA. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 6. Dose-response in microfluidic device for detecting activity of agents in reducing intraocular pressure in a glaucoma model. The dose-response behavior of colistin sulfate on intraocular pressure (IOP) was determined for its use as active agent in treating glaucoma. Colistin sulfate exhibited an EC50 of 0.36 nM for treatment of bovine vitreous humor.

The compound colistin sulfate was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ethanol or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

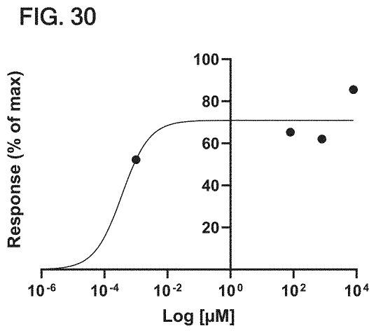

FIG. 30 shows the dose dependent response curve for the treatment of bovine vitreous humor glaucoma model with the compound colistin sulfate. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 7. Dose-response in microfluidic device for detecting activity of agents in reducing intraocular pressure in a glaucoma model. The dose-response behavior of polymyxin B sulfate on intraocular pressure (TOP) was determined for its use as active agent in treating glaucoma. Polymyxin B sulfate exhibited an EC50 of 4.3 nM for treatment of bovine vitreous humor glaucoma model.

The compound polymyxin B sulfate was tested in bovine vitreous humor (BVH) glaucoma model in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ethanol or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

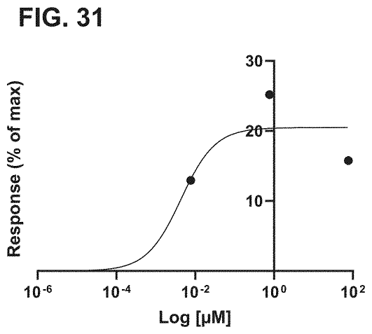

FIG. 31 shows the dose dependent response curve for the treatment of bovine vitreous humor glaucoma model with the compound polymyxin B sulfate. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 8. Isolation and extraction of extracellular matrix bodies in a microfluidic device. A microfluidic device was used to isolate bovine vitreous extracellular matrix bodies. After isolation, the bodies were extracted.

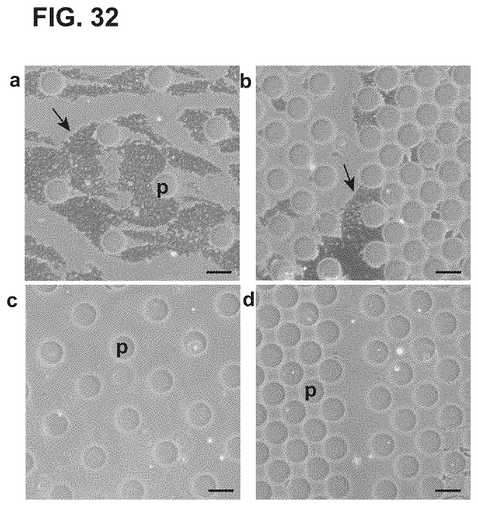

FIG. 32 shows isolation of extracellular matrix bodies in a restriction channel of a microfluidic device of this disclosure. FIG. 32a and FIG. 32a show representative photomicrographs of a microfluidic chip perfused with bovine vitreous humor. The letter “p” marks a pillar in the channel. After perfusion, extracellular matrix bodies were isolated between and around the pillars. FIG. 32c and FIG. 32d show representative photomicrographs of the channels after extracting extracellular matrix bodies. Extracellular matrix bodies were extracted from the chip using a mild detergent, 1% sodium dodecyl sulfate, SDS, and flow reversal out of the inlet port. Extracellular matrix bodies were dislodged from the chip, which showed substantially fewer bodies after extraction.

Example 9. Detection of biomarkers for extracellular matrix bodies by proteomic profile. Bovine vitreous extracellular matrix bodies were isolated and analyzed for their proteomic profile off-chip using LC/MS.

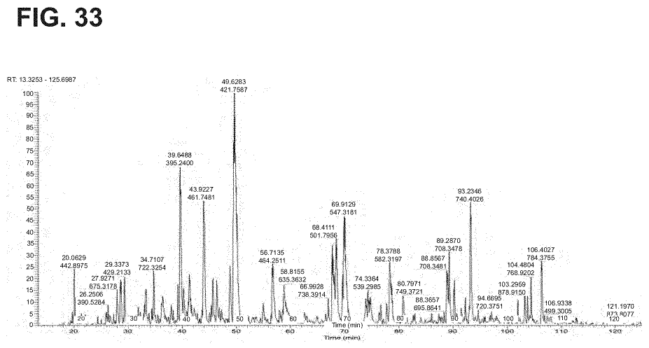

FIG. 33 shows proteomic analysis off-chip of bovine extracellular matrix bodies by LC/MS. Biomarkers for extracellular matrix bodies were detected.

A concentrated bovine vitreous extracellular matrix aggregate pellet was resuspended in 50 μl of 1% sodium dodecyl sulfate (SDS, Sigma), and pelleted again using 25 Kg centrifugation for 10 min at room temperature. The pellet was solubilized in 20 μl of 2× SDS, 50 mM dithiothreitol (DTT) reducing agent, sonicated for 10 min and incubated at 95° C. for 5 min. Both pellet and supernatant were subjected to electrophoresis into NuPAGE 10% Bis-Tris Gel (1.5 mm×10 well, Invitrogen). The gel was stained with Coomassie brilliant Blue R250. A photograph of the gel was captured, and stored, then the gel was de-stained for further analysis.

Each gel band was subjected to reduction with 10 mM DTT for 30 min at 60° C., alkylation with 20 mM iodoacetamide for 45 min at room temperature in the dark. The samples were digested with 0.2 μg trypsin (sequencing grade, Thermo Scientific Cat #90058), and incubated for 16 h at 37° C. Peptides were extracted twice with 5% formic acid, 60% acetonitrile and dried under vacuum.

Samples were analyzed by LC-MS using Nano LC-MS/MS (Dionex Ultimate 3000 RLSCanon System, Thermofisher) interfaced with Eclipse (ThermoFisher). 3 μl out of 12.5 μl of in-gel digested Sample Pellet was loaded on to a fused silica trap column (Acclaim PepMap 100, 75 um×2 cm, ThermoFisher). After washing for 5 min at 5 μl/min with 0.1% Trifluoroacetic acid (TFA), the trap column was brought in-line with an analytical column (Nanoease MZ peptide BEH C18, 130A, 1.7 μm, 75 μm×250 mm, Waters) for LC-MS/MS. Peptides were fractionated at 300 nL/min using a segmented linear gradient 4-15% B in 30 min (where A: 0.2% formic acid, and B: 0.16% formic acid, 80% acetonitrile), 15-25% B in 40 min, 25-50% B in 44 min, and 50-90% B in 11 min. Solution B then returns at 4% for 5 minutes for the next run.

The scan sequence began with an MS1 spectrum (Orbitrap analysis, resolution 120,000, scan range from M/Z 375-1500, automatic gain control (AGC) target 1E6, maximum injection time 100 ms). The top S (3 sec) duty cycle scheme were used for determining the number of MSMS performed for each cycle. Parent ions of charge 2-7 were selected for MSMS and dynamic exclusion of 60 s was used to avoid repeat sampling. Parent masses were isolated in the quadrupole with an isolation window of 1.2 m/z, automatic gain control (AGC) target 1E5, and fragmented with higher-energy collisional dissociation with a normalized collision energy of 30%. The fragments were scanned in Orbitrap with resolution of 15,000. The MSMS scan ranges were determined by the charge state of the parent ion but lower limit was set at 110 amu.

Selected extracellular matrix-associated proteins expressed in the vitreous bovine extracellular matrix bodies fraction were identified by proteomics profiling and are shown in Table 1.

TABLE 1
Extracellular matrix proteins in vitreous bovine extracellular matrix bodies

| | | | Spectral | |
|---|---|---|---|---|
| Description | Gene | ID | count | Function |
| Fibrillin-1 | FBN1 | A0A3Q1M7S1 | 129 | Fibrillin-1 is a large extracellular |
| | | | | matrix glycoprotein which assembles |
| | | | | to form 10-12 nm microfibrils in |
| | | | | extracellular matrix. |
| Fibulin-2 | FBLN2 | E1BEB4 | 107 | Fibulin-2 (FBLN2) is a secreted |
| | | | | extracellular matrix glycoprotein |
| | | | | which has been associated with tissue |
| | | | | development and remodeling. |
| Alpha-2- | Alpha-2-M | Q7SIH1 | 77 | Alpha2-macroglobulin (a2M) |
| macroglobulin | | | | secreted by tissue macrophages and |
| | | | | fibroblasts functions in the |
| | | | | environment of extracellular matrix |
| | | | | macromolecules. |
| Collagen | CO2A1 | P02459 | 74 | Collagen II gene encodes the alpha-1 |
| alpha-1(II) | | | | chain of type II collagen, a fibrillar |
| chain | | | | collagen found in cartilage and |
| | | | | the vitreous humor of the eye. |
| Clusterin | CLUS | P17697 | 74 | Functions as extracellular chaperone |
| | | | | that prevents aggregation of |
| | | | | nonnative proteins. |
| Spondin-1 | Q9GLX9 | Q9N0H5 | 38 | Spondin 1 (SPON1) is an ECM |
| | | | | protein primarily studied for its role |
| | | | | in nervous system development as a |
| | | | | nerve outgrowth signaling molecule. |
| Opticin | OPTC | P58874 | 37 | Opticin binds collagen fibrils; |
| | | | | Belongs to the small leucine-rich |
| | | | | proteoglycan (SLRP) family. |
| Contactin-1 | CNTN1 | Q28106 | 36 | Contactins mediate cell surface |
| | | | | interactions during nervous system |
| | | | | development. |
| Versican Core | CSPG2 | P81282 | 33 | Intercellular signaling and in |
| Protein | | | | connecting cells with the |
| | | | | extracellular matrix. |
| Myosin-10 | MYH10 | Q27991 | 26 | Cellular myosin that appears to play a |
| | | | | role in cytokinesis, cell shape, and |
| | | | | specialized functions such as |
| | | | | secretion and capping. |

The vitreous fraction was obtained by low-speed centrifugation and isolation using a microfluidic device. Higher spectral count values represent a greater amount of protein.

Selected proteins known to be involved in protein-aggregation found in the vitreous bovine extracellular matrix bodies fraction were identified by proteomics profiling and are shown in Table 2.

TABLE 2
Protein-aggregation proteins in vitreous bovine extracellular matrix bodies

| | | | Spectral | |
|---|---|---|---|---|
| Description | Gene | ID | count | Function |
| Complement C3 | C3 | Q2UVX4 | 408 | Complement C3 plays a central |
| | | | | role in the activation of the |
| | | | | complement system. After |
| | | | | activation C3b can bind |
| | | | | covalently, via its reactive |
| | | | | thioester, to cell surface |
| | | | | carbohydrates or immune |
| | | | | aggregates |
| Alpha-enolase | ENO1 | Q9XSJ4 | 151 | Enolase acts as a plasminogen |
| | | | | receptor and mediates the |
| | | | | activation of plasmin and |
| | | | | extracellular matrix |
| | | | | degradation. |
| Prostaglandin-H2 | PGH2 | O02853 | 74 | Catalyzes the conversion of |
| D-isomerase | | | | PGH2 to PGD2. |
| Clusterin | CLUS | P17697 | 74 | Extracellular chaperone that |
| | | | | prevents aggregation of |
| | | | | nonnative proteins. |
| Complement Factor B | CFB | P81187 | 41 | Factor B alternate pathway of |
| | | | | the complement system is |
| | | | | cleaved by factor D into 2 |
| | | | | fragments. |

The vitreous ECM aggregate fraction was obtained by low-speed centrifugation and isolation using a prototype microfluidic device. The proteins were categorized by function and the highlighted proteins are known to play a role in extracellular matrix. For example, complement C3 (spectral count, 408), alpha-enolase (spectral count, 151), and clusterin (spectral count, 74) were found at relatively high spectral counts.

Example 10. Isolation and extraction of extracellular matrix bodies in a microfluidic device. A microfluidic device was used to isolate bovine vitreous extracellular matrix bodies. After isolation, the bodies were extracted.

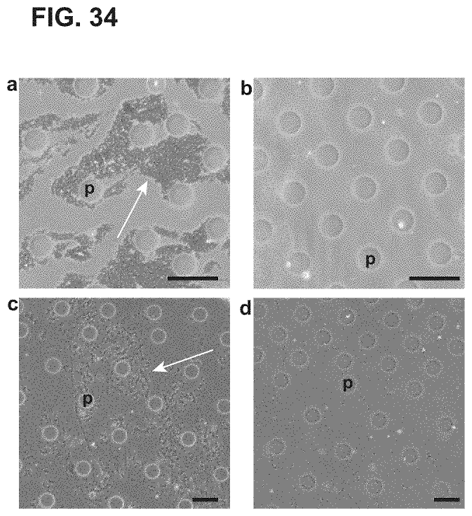

FIG. 34 shows isolation of extracellular matrix bodies in a restriction channel of a microfluidic device of this disclosure and their subsequent extraction. Image scale bars are 50 μm. FIG. 34a shows representative widefield photomicrographs of a microfluidic device perfused with bovine vitreous humor suspended in phosphate-buffered saline, pH 7.0 and counterstained for hyaluronic acid with alcian blue (grey signal, brightfield) FIG. 34a shows the signal from extracellular matrix bodies (arrows) trapped between the pillars (p) of the device. The chip was perfused with the biofluid for at least 60 minutes. After perfusion, the aggregates were isolated between pillars, observed in a mass-like formation. Material smaller than the extracellular matrix bodies material had exited via the outlet port. FIG. 34b shows extraction of extracellular matrix bodies from a restriction channel of a microfluidic device. The device was perfused with a mild detergent, 0.1% sodium dodecyl sulfate, SDS, and reversed flow direction from outlet to inlet. FIG. 34b exhibited substantially fewer bodies present in the channel after elution, and showed that the bodies were extracted. FIG. 34c shows a higher power image of extracellular matrix bodies (arrows) trapped between the pillars (p) after perfusion. FIG. 34c shows extraction with detergent and reverse flow, again showing substantially fewer bodies in the channel after extraction.

Example 11. Isolation and extraction of extracellular matrix bodies in a microfluidic device. This experiment showed that on-chip staining can be used to detect isolation of extracellular matrix bodies.

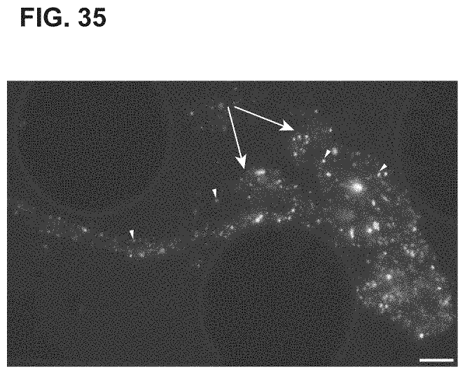

FIG. 35 shows on-chip immunohistochemical staining of extracellular matrix bodies in a device channel of this disclosure. The microfluidic device was infused with a fluid containing homogenized bovine vitreous suspended in a biofluid. After perfusion of the fluid into the device, the fluid flowed through the inlet and exited via the outlet. The larger extracellular matrix bodies (arrows) were trapped between the pillars (marked Lp). The chip was perfused with a blocking solution to prevent non-specific antibody binding before antibody staining. Next, protein fibronectin, a known extracellular matrix component and integrin-binding protein, was labeled by infusing anti-fibronectin primary antibody, incubating the sample for 2 hours, and washing. Then, goat anti-rabbit FITC secondary antibody was incubated for 1 hour and washed. The microfluidic chip was then imaged under wide-field fluorescence and brightfield microscopy. FIG. 35 shows a representative wide-field-fluorescent photomicrograph. This image shows extracellular matrix bodies in a microfluidic channel. The distance between large pillars (Lp) was about 100 μm. The punctate signal within the bodies represents fibronectin staining (anti-fibronectin Ab, goat anti-rabbit secondary antibody with Alexa 488, FITC, white signal).

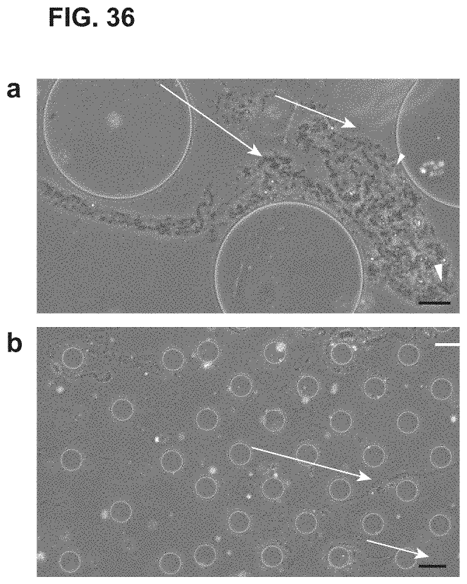

FIG. 36 shows on-chip immunohistochemical staining of extracellular matrix bodies in a device channel of this disclosure. FIG. 36a shows a representative photomicrograph brightfield image of the stained extracellular matrix bodies in a channel (arrows). Image scale bar was 20 μm. Control images had no fluorescent signal, which showed that the signal in FIG. 36a was specific for fibronectin. FIG. 36b again shows stained extracellular matrix bodies in a channel (arrow). Image scale bar was 50 μm. Again, control images had no fluorescent signal, which showed that the signal in FIG. 36b was specific for fibronectin.

Example 12. Isolation and extraction of extracellular matrix bodies in a microfluidic device. This experiment showed that on-chip staining can be used to detect isolation of extracellular matrix bodies.

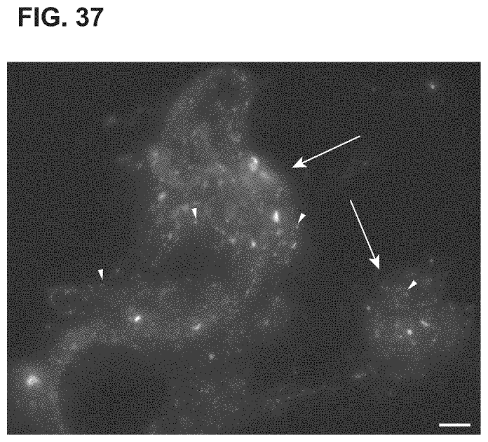

FIG. 37 shows on-chip immunohistochemical staining of extracellular matrix bodies in a device channel of this disclosure. FIG. 37 shows a representative photomicrograph of on-chip immunohistochemical staining of perlecan protein, a component of the extracellular matrix of cartilage, in a biofluid containing extracellular matrix bodies. The microfluidic chip was infused with a fluid containing homogenized bovine vitreous suspended in a biofluid. After perfusion of the biofluid into the device, the sample flowed through the inlet and exited via the outlet. The larger extracellular matrix bodies (arrows) were trapped between pillars (marked “p”). The chip was perfused with a blocking solution to prevent non-specific antibody binding before antibody staining. Perlecan was labeled by infusing anti-perlecan primary antibody, incubating the sample for 2 hours, and washing. Then, goat anti-rabbit TRITC secondary antibody was incubated for 1 hour and washed. The microfluidic chip was then imaged under wide-field fluorescence and brightfield microscopy. FIG. 37 shows extracellular matrix bodies in a microfluidic channel between pillars (p). The punctate signal represents perlecan staining (white signal). Control images had no fluorescent signal, which showed that the signal in FIG. 37 was specific for perlecan.

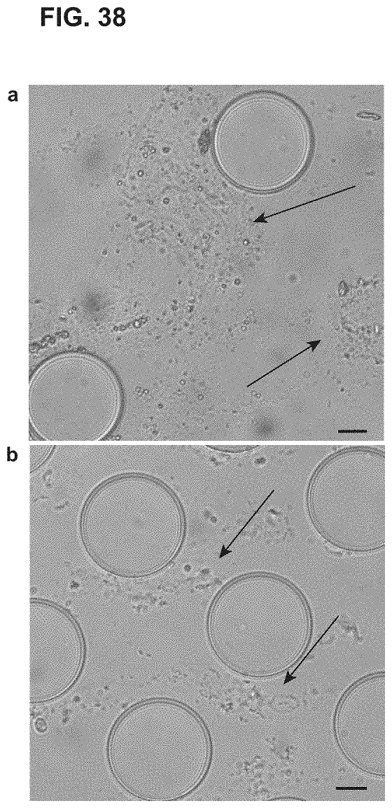

FIG. 38 shows on-chip immunohistochemical staining of extracellular matrix bodies in a device channel of this disclosure. FIG. 38a shows a representative photomicrograph brightfield image of on-chip immunohistochemical staining of perlecan. Control images had no fluorescent signal, which showed that the signal in FIG. 38a was specific for perlecan. Image scale bar was 10 μm. FIG. 38b shows a representative photomicrograph brightfield image of on-chip immunohistochemical staining of perlecan. Control images had no fluorescent signal, which showed that the signal in FIG. 38c was specific for perlecan. Image scale bar was 10 μm.

Example 13. Off-chip analysis of extracellular matrix bodies extracted from a microfluidic device. This experiment showed off-chip analysis of extracellular matrix bodies as can be extracted from a device channel of this disclosure.

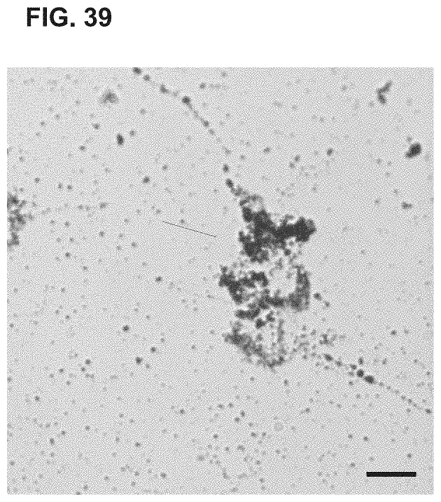

FIG. 39 shows extracellular matrix bodies can be visualized on a glass surface using 1-Ethyl-3-(3-dimethylaminopropyl) carbodiimide crosslinking and staining of hyaluronic acid with alcian blue dye. FIG. 39 shows a signal (dark stain) for extracellular matrix bodies, which showed that EDC crosslinking retained the extracellular matrix bodies on the surface.

Example 14. Off-chip analysis of extracellular matrix bodies extracted from a microfluidic device. This experiment showed off-chip analysis of extracellular matrix bodies of this disclosure.

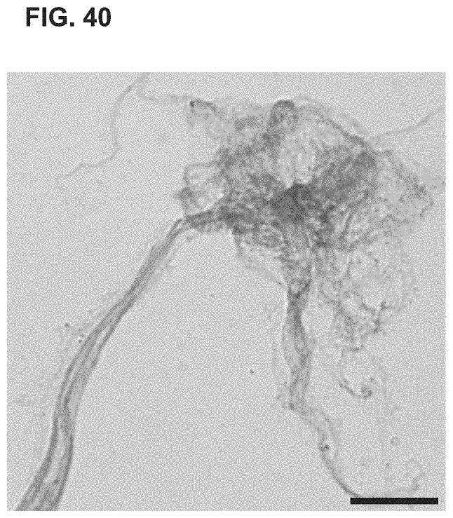

FIG. 40 shows off-chip analysis of extracellular matrix that can be visualized on a glass surface using 1-Ethyl-3-(3-dimethylaminopropyl) carbodiimide crosslinking and staining of collagen using picrosirius red dye. FIG. 40 shows a signal (dark stain) for collagen strands within extracellular matrix bodies, which showed that EDC crosslinking retained the extracellular matrix bodies on the surface. This also shows EMB can be visualized by collagen staining.

Example 15. Off-chip analysis of extracellular matrix bodies extracted from a microfluidic device. This experiment showed off-chip analysis of extracellular matrix bodies that can be visualized on a glass surface with a nucleic acid marker.

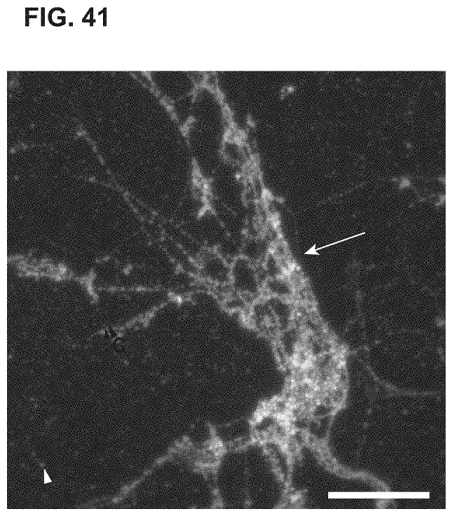

FIG. 41 shows off-chip analysis of extracellular matrix bodies that can be visualized on a glass surface using 1-Ethyl-3-(3-dimethylaminopropyl) carbodiimide crosslinking and staining of DNA using Hoechst dye. FIG. 41 shows signal for DNA in extracellular matrix bodies.

Example 16. Isolation and detection of extracellular matrix bodies in a microfluidic device. This experiment showed isolation and detection of extracellular matrix bodies in a device channel of this disclosure.

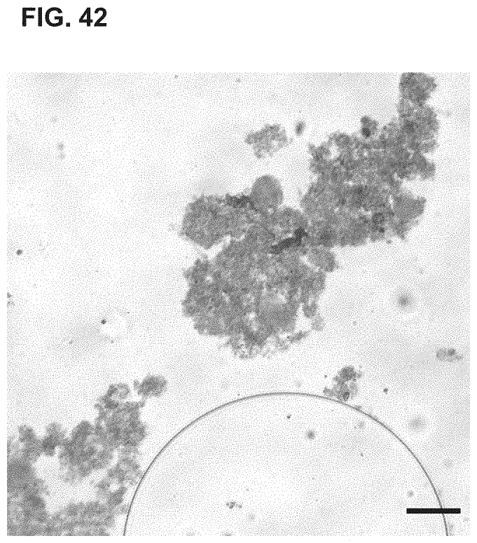

FIG. 42 shows on-chip isolation and detection of extracellular matrix bodies. FIG. 42 shows a representative photomicrograph of a microfluidic chip perfused with bovine vitreous humor suspended in the phosphate-buffered saline, pH 7.0, and counterstained for hyaluronic acid with alcian blue, dark stain, brightfield. FIG. 42 shows signal from extracellular matrix bodies trapped near a large pillar (circle) of the device. The chip was perfused with the biofluid for at least 60 minutes. Extracellular matrix bodies were observed in a cluster-like formation. Image scale bar was 50 μm.

Example 17. Isolation, detection and analysis of extracellular matrix bodies on-chip in a microfluidic device. This experiment showed isolation, detection and analysis of extracellular matrix bodies on-chip in a device channel of this disclosure.

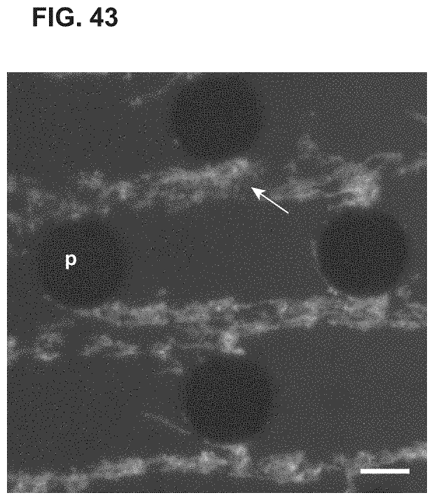

FIG. 43 shows on-chip isolation, detection and analysis of extracellular matrix bodies in a microfluidic device. FIG. 43 shows a representative low-power fluorescent photomicrograph of a channel after perfusion of bovine vitreous humor extracellular matrix bodies that were counterstained for protein with carboxyfluorescein succinimidyl ester (CFSE). The microfluidic chip was infused with a fluid containing homogenized bovine vitreous. After perfusion of the fluid into the device, the fluid flowed through the inlet and exited via the outlet. The larger extracellular matrix bodies were trapped near pillars (marked “p”). Image scale bar was 25 μm.

Example 18. Isolation, detection and analysis of extracellular matrix bodies on-chip in a microfluidic device. This experiment showed isolation, detection and analysis of extracellular matrix bodies on-chip in a device channel of this disclosure.

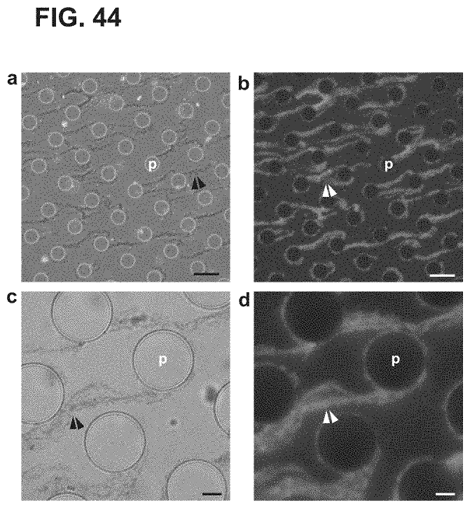

FIG. 44 shows on-chip isolation, detection and analysis of extracellular matrix bodies in a microfluidic device. FIG. 44 shows a representative photomicrograph of a microfluidic chip perfused with bovine vitreous humor suspended in the phosphate-buffered saline, pH 7.0, and counterstained for collagen with picrosirius red, dark stain, brightfield. FIG. 44a shows signal from extracellular matrix bodies between pillars (marked “p’) of the device. The chip was perfused with the biofluid for at least 60 minutes. FIG. 44b shows the same image with a fluorescent filter, and shows that collagen was detected with picrosirius red (light grey signal). Image scale bar was 50 μm. FIG. 44c and FIG. 44c show the images at a higher power. Image scale bar was 10 μm

Example 19. Isolation, detection and analysis of extracellular matrix bodies with a microfluidic device. This experiment showed isolation, detection and analysis of extracellular matrix bodies with a device of this disclosure.

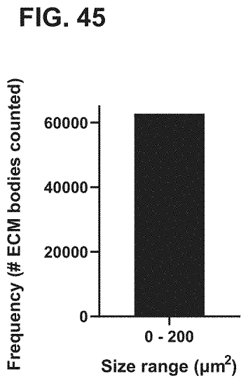

FIG. 45 shows frequency size distribution of human extracellular matrix bodies present in human aqueous humor biofluids from healthy and pre-disease states, glaucoma suspect and pre-glaucoma. Aqueous humor was obtained from 8 patients, healthy sample or pre-glaucoma diagnosis, having intraocular pressures ranging from 9 to 25 mmHg. The human samples were not processed by centrifugation or other means. The size of extracellular matrix bodies was determined by crosslinking sample to a glass slide using a carbodiimide EDC fixative, staining with uranyl acetate, and imaging with wide-field microscopy. The size was quantified using an automated program (ImageJ) in all eight samples. The size (area) of extracellular matrix bodies ranged from about 1.67 μm² to about 67×10³ μm². FIG. 45 shows the count of extracellular matrix bodies in the 0-200 μm² range.

Example 20. Isolation, detection and analysis of extracellular matrix bodies with a microfluidic device. This experiment showed isolation, detection and analysis of extracellular matrix bodies with a device of this disclosure.

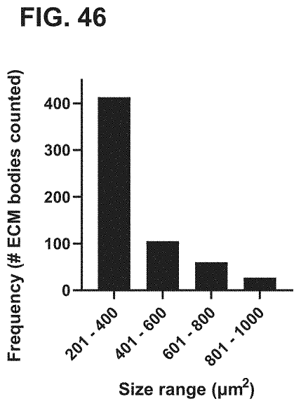

FIG. 46 shows frequency size distribution of human extracellular matrix bodies present in human aqueous humor biofluids from healthy and pre-disease states, glaucoma suspect and pre-glaucoma. Aqueous humor was obtained from 8 patients, healthy sample or pre-glaucoma diagnosis, having intraocular pressures ranging from 9 to 25 mmHg. The human samples were not processed by centrifugation or other means. The size of extracellular matrix bodies was determined by crosslinking sample to a glass slide using a carbodiimide EDC fixative, staining with uranyl acetate, and imaging with wide-field microscopy. The size was quantified using an automated program (ImageJ) in all eight samples. The size (area) of extracellular matrix bodies ranged from about 1.67 μm² to about 67×10³ μm². FIG. 46 shows the count of extracellular matrix bodies in the 201-1000 μm² range.

Example 21. Isolation, detection and analysis of extracellular matrix bodies with a microfluidic device. This experiment showed isolation, detection and analysis of extracellular matrix bodies with a device of this disclosure.

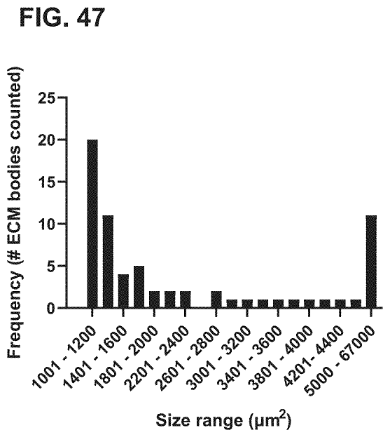

FIG. 47 shows frequency size distribution of human extracellular matrix bodies present in human aqueous humor biofluids from healthy and pre-disease states, glaucoma suspect and pre-glaucoma. Aqueous humor was obtained from 8 patients, healthy sample or pre-glaucoma diagnosis, having intraocular pressures ranging from 9 to 25 mmHg. The human samples were not processed by centrifugation or other means. The size of extracellular matrix bodies was determined by crosslinking sample to a glass slide using a carbodiimide EDC fixative, staining with uranyl acetate, and imaging with wide-field microscopy. The size was quantified using an automated program (ImageJ) in all eight samples. The size (area) of extracellular matrix bodies ranged from about 1.67 μm² to about 67×10³ μm². FIG. 47 shows the count of extracellular matrix bodies in the 1001-5000 μm² range.

Example 22. Isolation, detection and analysis of extracellular matrix bodies with a microfluidic device. This experiment showed isolation, detection and analysis of extracellular matrix bodies with a device of this disclosure.

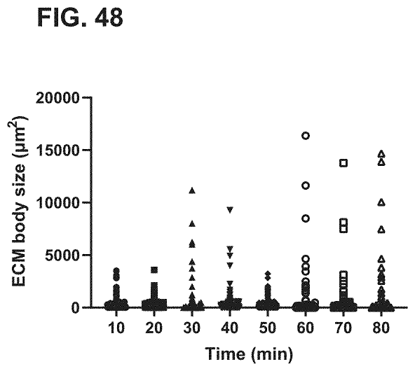

FIG. 48 shows size distribution of bovine vitreous extracellular matrix bodies isolated and extracted from a microfluidic device of this invention. Extracellular matrix bodies in bovine vitreous humor biofluid after isolation and extraction from a microfluidic device of this invention. The chip was perfused with the biofluid for at least 60 minutes. After 60 minutes of perfusion, the ECM bodies were isolated near restriction channel pillars. Next, the chip was treated with a detergent (0.1% sodium dodecyl sulfate, SDS), and the sample extracted from the chip via reverse flow, which allowed the bodies to flow out of the inlet port. Fractions of the eluate were collected at 10-minute intervals for a total of 80 minutes. Sample was mounted on a glass slide and stained with alcian blue, and imaged with wide-field microscopy. The size was quantified using an automated program (ImageJ). The size (area) of extracellular matrix bodies was up to about 16×10³ μm². FIG. 48 shows the count of extracellular matrix bodies in each eluate fraction, which increased over time. This experiment showed that a microfluidic device of this invention can be used to isolate and extract extracellular matrix bodies of various sizes.

Example 23. Isolation, detection and analysis of extracellular matrix bodies on-chip in a microfluidic device. This experiment showed isolation, detection and analysis of extracellular matrix bodies off-chip.

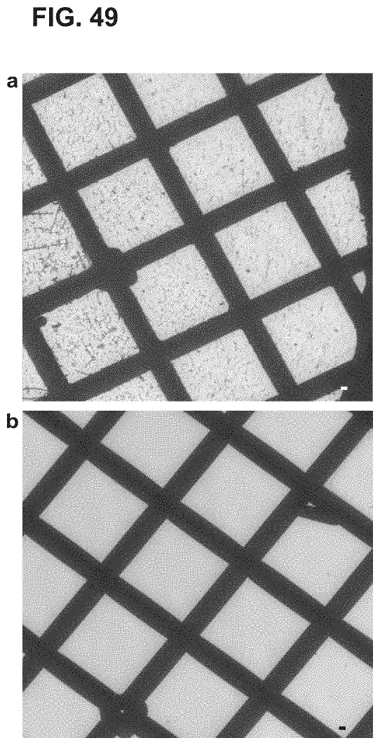

FIG. 49 shows for off-chip analysis of extracellular matrix bodies can be done with retained with 1-ethyl-3-(3-dimethylaminopropyl) carbodiimide (EDC) crosslinking. FIG. 49a shows a representative TEM image of extracellular matrix bodies from native bovine vitreous humor obtained with EDC crosslinking. Extracellular matrix bodies were observed with EDC fixation. FIG. 49b shows a similar image taken without EDC crosslinking. Extracellular matrix bodies were not observed without EDC fixation.

Example 24. Isolation and detection of extracellular matrix bodies in early-stage pancreatic cancer with a human plasma sample using a microfluidic device. This experiment showed isolation and detection of extracellular matrix bodies in early-stage pancreatic cancer with a human plasma sample using a microfluidic device. This experiment showed that extracellular matrix bodies in early-stage pancreatic cancer can be isolated and detected from a human plasma sample using a microfluidic device of this invention. This experiment further showed that extracellular matrix bodies are a useful marker for differentiation of early stage pancreatic ductal adenocarcinoma plasma from healthy controls.

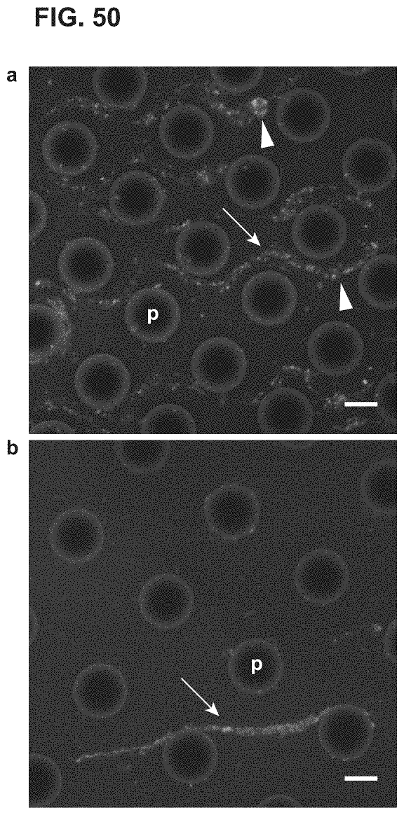

FIG. 50 shows isolation of extracellular matrix bodies from a human plasma sample from a patient with early-stage pancreatic ductal adenocarcinoma (PDAC) as compared to a healthy control.

In this experiment, a microfluidic chip was infused with human plasma from early-stage pancreatic ductal adenocarcinoma (PDAC). After perfusion into the device, the biofluid flowed through the inlet and exited via the outlet. Results were compared to a healthy age-matched control.

The chip was perfused with a blocking solution to prevent non-specific antibody binding before antibody staining. Next, protein fibronectin, a known extracellular matrix component, and integrin-binding protein, was labeled by on-chip immunohistochemical staining by infusing anti-fibronectin primary antibody, incubating the sample for 2 hours, and washing. Then, goat anti-rabbit FITC secondary antibody was incubated for 1 hour and washed. The microfluidic chip was then imaged under fluorescence microscopy.

FIG. 50a shows the representative wide-field-fluorescent photomicrograph image for the PDAC sample. FIG. 50a shows extracellular matrix bodies lodged in a microfluidic channel. FIG. 50a further shows the larger extracellular matrix bodies (arrows) were lodged between pillars (marked “p”). The width between pillars was about 100 μm. The punctate signal from the lodged extracellular matrix bodies represents fibronectin staining (anti-fibronectin Ab, goat anti-rabbit secondary Ab with Alexa 488, FITC, white signal). The staining showed an abundant signal and punctate staining within the EMB (FIG. 50a, arrowheads).

FIG. 50b shows a similarly-obtained fluorescent photomicrograph of an age-matched healthy control human plasma sample. FIG. 50b shows a markedly reduced amount of fibronectin signal (FIG. 50b, grey signal, arrow). The healthy control signal was far smaller than for the disease signal when processed under identical conditions. Image scale bars were 20 μm.

1.-56. (canceled)

57. A device for isolating a fraction of a biological sample, comprising:
one or more restriction channels having an inlet end and an outlet end, wherein the inlet end and outlet end are in fluid communication through the channel;
a plurality of spaced-apart obstructions lodged in the restriction channels for providing resistance to flow, wherein the spacing between obstructions decreases in the direction from the inlet end to the outlet end; and
an inlet reservoir for holding a fluid, wherein the inlet fluid reservoir is in fluid communication with the inlet end of the restriction channels;
one or more uniform flow channels having an inlet end and an outlet end, wherein the inlet end and outlet end are in fluid communication through the channel, wherein the inlet end is in fluid communication with the inlet reservoir.

58. The device of claim 1, further comprising
a pressure source for applying pressure to the fluid in the inlet reservoir;
a flow sensor in fluid communication with the inlet reservoir for measuring the flow rate and pressure of the fluid at the inlet reservoir.

59. The device of claim 1, further comprising an outlet reservoir in fluid communication with the outlet ends of the restriction channels and uniform flow channels.

60. The device of claim 1, wherein the restriction channels comprise a barrier band having fenestrations of at least about 1 micrometers, or at least about 2 micrometers, or at least about 4 micrometers, or at least about 10 micrometers, or at least about 25 micrometers, or at least about 50 micrometers, or at least about 100 micrometers, or at least about 200 micrometers, or at least about 500 micrometers.

61. The device of claim 1, wherein the restriction channels comprise fenestrations of about 1-4 micrometers, or about 1-15 micrometers, or about 4-35 micrometers, or about 4-100 micrometers, or about 4-200 micrometers.

62. The device of claim 1, wherein from 1-90% of the flow in the device is within uniform flow channels, or from 1-75% of the flow in the device is within uniform flow channels, or from 1-50% of the flow in the device is within uniform flow channels, or from 1-25% of the flow in the device is within uniform flow channels.

63. The device of claim 1, wherein the restriction channels and the uniform flow channels are integral with the same chip or substrate, or are in different chips or substrates.

64. The device of claim 1, wherein the restriction channels are microfluidic channels.

65. The device of claim 1, comprising means for analyzing the biological sample in the channels, means for analyzing a proteomic composition, a lipidomic composition, a transcriptomic composition, or a carbohydrate composition of the biological sample in the channels, means for measuring the level of the isolated fraction of the sample within the channels, or means for measuring the level of a biomarker in the isolated fraction of the sample within the channels.

66. The device of claim 1, wherein the plurality of obstructions comprise pillars integral with the channels.

67. The device of claim 1, wherein the plurality of obstructions comprise one or more of:
a portion of a human or animal uveal meshwork;
a portion of a human or animal corneoscleral meshwork; and
a portion of a human or animal juxtacanalicular meshwork.

68. The device of claim 1, wherein the plurality of obstructions comprise glass beads, magnetic beads, gel particles, dextran particles, or polymer particles.

69. The device of claim 1, wherein the biological sample is composed of human or animal bodily fluid, blood, tissue, or cells.

70. The device of claim 1, wherein the biological sample comprises a carrier fluid.

71. The device of claim 1, wherein the biological sample comprises one or more reagents.

72. The device of claim 1, wherein the restriction channels further comprise binding moieties for binding a biomarker or biomolecule of the sample.

73. The device of claim 1, wherein the biological sample is from a subject undergoing a diagnosis or prognosis.

74. The device of claim 1, further comprising a serpentine fluid mixing region in the restriction channels.

75. The device of claim 1, wherein the restriction channels or continuous flow channels have a fluorinated coating.

76. A microfluidic system for isolating a fraction of a biological sample, the system comprising:
a microfluidic device comprising
one or more restriction channels having an inlet end and an outlet end, wherein the inlet end and outlet end are in fluid communication through the channel;
a plurality of spaced-apart obstructions lodged in the restriction channels for providing resistance to flow, wherein the spacing between obstructions decreases in the direction from the inlet end to the outlet end; and
an inlet reservoir for holding a fluid, wherein the fluid reservoir is in fluid communication with the inlet end of the restriction channels; and
one or more uniform flow channels having an inlet end and an outlet end, wherein the inlet end and outlet end are in fluid communication through the channel, wherein the inlet end is in fluid communication with the inlet reservoir;
a drive unit comprising a pressure source;
a source unit comprising a fluid source, wherein the pressure source is in fluid communication with the fluid source and the inlet reservoir of the microfluidic device;
a sensor unit comprising a sensor in fluid communication with the inlet reservoir for measuring the flow rate and pressure of the fluid at the inlet reservoir and sending the flow and pressure data to a processor; and
an on-chip analyzer unit comprising one or more means for analyzing the isolated fraction in the microfluidic device and sending the analysis data to a processor; and
a processor for receiving and displaying the flow rate, pressure and analysis.
